SEQUENCE LISTING (53 sequences)

SEQ ID NO: 1 - DNA, 3045 bp, Escherichia coli
  source 1..3045: mol_type = other DNA
atgggtaaccaatggcaacaaaaatatcttcttgagtacaatgagttggtatcaaatttcccttcacctgaaagagttgtcagcgattacattaagaattgttttaaaactgacttgccgtggtttagtcggattgatcctgataatgcttatttcatctgcttttctcaaaaccggagtaatagcagatcttatactggatgggatcatcttgggaaatataaaacagaagtactgacactcactcaagccgctcttattaatattggttatcgttttgatgtttttgatgatgcaaattcaagcacaggaatttataaaacaaagagtgcagatgtgtttaacgaagaaaatgaagaaaaaatgctcccgtcggaatacctgcattttttacaaaagtgtgattttgcaggtgtttatggaaaaactctgtcagattactggtcgaaatactatgataaatttaagcttttactaaaaaattattatatttcttctgctttgtatctttataaaaatggagagcttgatgagcgtgaatataatttctccatgaacgccttaaatcgcagtgataatatatcactattattctttgatatttatggatattacgcatctgatatttttgtagccaaaaataatgataaggtaatgcttttcattcctggtgcaaaaaaaccttttttattcaagaagaatatcgctgatttgcggcttacccttaaagaacttattaaggatagtgacaaacaacaattactttcccaacatttttcattatatagtcgtcaagatggagtttcctatgcaggagtaaattctgttctacatgcaatagaaaatgatggtaattttaatgagtcttactttctgtattccaataagacacttagcaataaagatgtttttgatgctatagctatttctgttaagaaacgcagtttcagtgatggtgatatcgttataaaatcaaacagtgaagctcaacgagactatgctctgactatactccagacgattttatcaatgacccctatatttgatatcgtagtcccggaggtatctgttccgcttggactggggattattacttccagtatggggatcagttttgatcaactgattaatggtgatacttatgaagaacgtcgttctgctatacctggtttggcgacaaatgcagtattgcttggtctgtcttttgcaattccactcttgattagtaaggcaggaataaaccaggaggtacttagcagcgttataaataatgagggcaggactctgaatgaaacaaatatcgatatatttttgaaggaatatggaattgctgaagatagtatatcctcaactaatttgttagacgttaagcttaaaagttccgggcagcatgtcaatattgtaaagcttagtgatgaagataatcaaattgtcgctgtaaaagggagttctctgagcggcatctactatgaagtggacattgaaacaggatatgagattttatcccgaagaatttatcgtaccgaatataataatgaaattctctggactcgaggtggtggtctaaaaggggggcagccatttgattttgaaagtctcaatattcctgtattttttaaagatgaaccctattctgcagtgaccggatctccgttatcatttattaatgatgacagctcacttttatatcctgatacaaacccaaaattaccgcaaccaacgtcagaaatggatattgttaattatgttaagggttctggaagctttggggatagatttgtaactttgatgagaggagctactgaggaagaagcatggaatattgcctcttatcatacggctgggggaagtacagaagaattacacgaaattttgttaggtcagggcccacagtcaagcttaggttttactgaatatacctcaaatgttaacagtgcagatgcagcaagcagacgacactttctggtagttataaaagtgcacgtaaaatatatcaccaataataatgtttcatatgttaatcattgggcaattcctgatgaagccccggttgaagtactggctgtggttgacaggagatttaattttcctgagccatcaacgcctcctgatatatcaaccatacgtaaattgttatctctacgatattttaaagaaagtatcgaaagcacctccaaatctaactttcagaaattaagtcgcggtaatattgatgtgcttaaaggacggggaagtatttcatcgacacgtcagcgtgcaatctatccgtattttgaagccgctaatgctgatgagcaacaacctctctttttctacatcaaaaaagatcgctttgataaccatggctatgatcagtatttctatgataatacagtggggctaaatggtattccaacattgaacacctatactggggaaattccatcagactcatcttcactcggctcaacttattggaagaagtataatcttactaatgaaacaagcataattcgtgtgtcaaattctgctcgtggggcgaatggtattaaaatagcacttgaggaagtccaggagggtaaaccagtaatcattacaagcggaaatctaagtggttgtacgacaattgttgcccgaaaagaaggatatatttataaggtacatactggtacaacaaaatctttggctggatttaccagtactaccggggtgaaaaaagcagttgaagtacttgagctacttacaaaagaaccaatacctcgcgtggagggaataatgagcaatgatttcttagtcgattatctgtcggaaaattttgaagattcattaataacttactcatcatctgaaaaaaaaccagatagtcaaatcactattattcgtgataatgtttctgttttcccttacttccttgataatatacctgaacatggctttggtacatcggcgactgtactggtgagagtggacggcaatgttgtcgtaaggtctctgtctgagagttattctctgaatgcagatgcctccgaaatatcggtattgaaggtattttcaaaaaaattttga

SEQ ID NO: 2 - protein, 1014 aa, Escherichia coli
MGNQWQQKYLLEYNELVSNFPSPERVVSDYIKNCFKTDLPWFSRIDPDNAYFICFSQNRSNSRSYTGWDHLGKYKTEVLTLTQAALINIGYRFDVFDDANSSTGIYKTKSADVFNEENEEKMLPSEYLHFLQKCDFAGVYGKTLSDYWSKYYDKFKLLLKNYYISSALYLYKNGELDEREYNFSMNALNRSDNISLLFFDIYGYYASDIFVAKNNDKVMLFIPGAKKPFLFKKNIADLRLTLKELIKDSDKQQLLSQHFSLYSRQDGVSYAGVNSVLHAIENDGNFNESYFLYSNKTLSNKDVFDAIAISVKKRSFSDGDIVIKSNSEAQRDYALTILQTILSMTPIFDIVVPEVSVPLGLGIITSSMGISFDQLINGDTYEERRSAIPGLATNAVLLGLSFAIPLLISKAGINQEVLSSVINNEGRTLNETNIDIFLKEYGIAEDSISSTNLLDVKLKSSGQHVNIVKLSDEDNQIVAVKGSSLSGIYYEVDIETGYEILSRRIYRTEYNNEILWTRGGGLKGGQPFDFESLNIPVFFKDEPYSAVTGSPLSFINDDSSLLYPDTNPKLPQPTSEMDIVNYVKGSGSFGDRFVTLMRGATEEEAWNIASYHTAGGSTEELHEILLGQGPQSSLGFTEYTSNVNSADAASRRHFLVVIKVHVKYITNNNVSYVNHWAIPDEAPVEVLAVVDRRFNFPEPSTPPDISTIRKLLSLRYFKESIESTSKSNFQKLSRGNIDVLKGRGSISSTRQRAIYPYFEAANADEQQPLFFYIKKDRFDNHGYDQYFYDNTVGLNGIPTLNTYTGEIPSDSSSLGSTYWKKYNLTNETSIIRVSNSARGANGIKIALEEVQEGKPVIITSGNLSGCTTIVARKEGYIYKVHTGTTKSLAGFTSTTGVKKAVEVLELLTKEPIPRVEGIMSNDFLVDYLSENFEDSLITYSSSEKKPDSQITIIRDNVSVFPYFLDNIPEHGFGTSATVLVRVDGNVVVRSLSESYSLNADASEISVLKVFSKKF

SEQ ID NO: 3 - DNA, 3045 bp, Escherichia coli
  source 1..3045: mol_type = other DNA
atgaacgttcaatggcaacaaaaatatcttcttgagtacaatgagttggtatcaaatttcccttcgccagaaagagtagttagcgattacattaggcgttgtttcaaaacagacttaccttggtttagtcaagtggatcctgataacacttattttatacgattttctcaaagtcggagtaatagtagatcttacaccggatgggatcatcttggaaaatataaaacaggagtactgacactcactcaagccgcacttattaatattggctatcattttgatgtttttgatgatgcaaatgcaagtgcgggaatctataaaacaagcagtgcagatatgtttaacgaaaaaaatgaagagaaaatgcttccttcagaatacctttattttttgaaagggtgtgatttttcaggtatttatggtcgatttctgtcagattattggtcgaaatactatgataaatttaagttgttactaaaaaattattatatttcctcagctttatatctttataaaaacggagaaattgatgaatatgaatataatttctctattagcgcattgaatcgtagagataatatatcattattcttctttgatatctatgggtattactcctctgatatgtttgtggccaagaacaatgaacgggtaatgctttttattcctggcgcaaaaaaaccttttttattcgaaaaaaatattgctgatttacgaattagtcttaaaaatctaattaaagagaacgacaataaacagttactatcccagcatttttcgttatatagtcgtcaagatggaattacttatgcgggtgtaaattcagtgctcaatgcaatagaaaatgatggcgtttttaatgaatcttactttctgtactccaataaaaggattaataataaagatgttttcgatgccgtagcattttctgtcaagaaacgtagttttagtgatggtgatatcgttataaaatcaaacagtgaggctcaacgagattatgcactgaccatacttcagactattttatcaatgactcctatatttgatgtcgcaattccggaggtgtctgttacgcttggactggggattattgcttccagtatggggatcagttttgatcagttaattaatggtgatacttatgaagaacgtcgttctgctatccctggtttagccacaaatgcagcattgcttgggttatcttttgcaattccgtttttgattagtaaggcgggcacaaaccagaagatacttagtagatatacaaagcatgagatcaggactctgaatgaaacaaatatcgatatgtttttggaggaatatggaattaataaaaatagtatatcagaaactaaagtattagaagttgagcttaaaggttctgggcagcatgtcaatattgtaaagcttagtgatgaagatagtaaaatcgtcgctgtaaaagggaattctctgagcggcatctactatgaagttgacattgaaacaggatatgaaatttcatccagaagaatttatcgtaccgaatacaatgataaaattttctggactcgaggtggtgggcttaagggggggcaatcgttcgattttgaaagtctcaaactccctatatttttcaaagatgaaccatattctgcagtaccggggtcttcgttatcatttattaatgatgatagctctcttttatatcctaattcgaccccaaaactaccgcaaccaacgccagaaatggagattgttaattacgttaaaagagctggagactttggggagagacttgtaaccttgatgcgaggaacaactgaagaagaagcgtggaatattgcccgttatcatacggcaggaggaagcaccgaagaattgcatgagattttgttggggcaaggcccacagtcgagtttaggttttactgaatatacttcaaatattaacagtgcagatgcggcaagcagacgacattttcttgtagtcataaaagtgcaagtgaaatatataaacaataataacgtttcgcatgttaatcactgggcaattcctgatgaggctccagtagaagtactggctgtggttgacaggagatttaatttccctgagccatcaactccacctaatatatcaattatacacaagttgttatctctgagatattttaaagaaaatatcgaaagtacatcaaggcttaacttacagaaattaaatcgtggtaatattgatatatttaaagggagggggagtatttcatcaacacgtcagcgtgcgatttatccgtattttgaatctgctaatgctgatgagcaacaacctgtctttttctacatcaaaaaaaaccggtttgatgactttggctatgatcaatatttctataatagtacagtggggttgaatggtattcccacattgaacacctatactggagaaattctatcagacgcatcctcgctcggctcaacttattggaagaagtataatctcactaatgaaacaagcatcattcgtgtatcaaattctgctcgaggggcaaatggtataaaaatagcacttgaagaagtgcaggaaggtaaaccggtaatcattacaagcggaaatttgagcggttgtacaacaattgttgctcgaaaaggaggatacctttataaggtacatacaggtacaacaatacctttagctggttttacaagtacaacaggggtaaaaaaagctgtagaagtttttgaattacttacaaataatccaatgccgcgcgtagagggagtaatgaataatgattttttggtaaattatctggcggaaagttttgatgagtctttaataacgtactcatcatctgaacaaaaaataggtagtaagattactatttctcgcgacaatgtttctacttttccttactttcttgataacataccagaaaaaggctttggtacatcggtgactatattggtaagagtagatggtaatgttatcgtaaaatccttatctgagagttattctttaaatgtagaaaactccaatatatcagtattgcatgttttttcaaaagatttttga

SEQ ID NO: 4 - protein, 1014 aa, Escherichia coli
MNVQWQQKYLLEYNELVSNFPSPERVVSDYIRRCFKTDLPWFSQVDPDNTYFIRFSQSRSNSRSYTGWDHLGKYKTGVLTLTQAALINIGYHFDVFDDANASAGIYKTSSADMFNEKNEEKMLPSEYLYFLKGCDFSGIYGRFLSDYWSKYYDKFKLLLKNYYISSALYLYKNGEIDEYEYNFSISALNRRDNISLFFFDIYGYYSSDMFVAKNNERVMLFIPGAKKPFLFEKNIADLRISLKNLIKENDNKQLLSQHFSLYSRQDGITYAGVNSVLNAIENDGVFNESYFLYSNKRINNKDVFDAVAFSVKKRSFSDGDIVIKSNSEAQRDYALTILQTILSMTPIFDVAIPEVSVTLGLGIIASSMGISFDQLINGDTYEERRSAIPGLATNAALLGLSFAIPFLISKAGTNQKILSRYTKHEIRTLNETNIDMFLEEYGINKNSISETKVLEVELKGSGQHVNIVKLSDEDSKIVAVKGNSLSGIYYEVDIETGYEISSRRIYRTEYNDKIFWTRGGGLKGGQSFDFESLKLPIFFKDEPYSAVPGSSLSFINDDSSLLYPNSTPKLPQPTPEMEIVNYVKRAGDFGERLVTLMRGTTEEEAWNIARYHTAGGSTEELHEILLGQGPQSSLGFTEYTSNINSADAASRRHFLVVIKVQVKYINNNNVSHVNHWAIPDEAPVEVLAVVDRRFNFPEPSTPPNISIIHKLLSLRYFKENIESTSRLNLQKLNRGNIDIFKGRGSISSTRQRAIYPYFESANADEQQPVFFYIKKNRFDDFGYDQYFYNSTVGLNGIPTLNTYTGEILSDASSLGSTYWKKYNLTNETSIIRVSNSARGANGIKIALEEVQEGKPVIITSGNLSGCTTIVARKGGYLYKVHTGTTIPLAGFTSTTGVKKAVEVFELLTNNPMPRVEGVMNNDFLVNYLAESFDESLITYSSSEQKIGSKITISRDNVSTFPYFLDNIPEKGFGTSVTILVRVDGNVIVKSLSESYSLNVENSNISVLHVFSKDF

SEQ ID NO: 5 - DNA, 3040 bp, Escherichia coli
  source 1..3040: mol_type = other DNA
atgaatacccaatggcaacaaaaataccttctggaatataatgatctggtgtcaaaatttccgtcaccagagaaagtaaccagcgactatattaagcataaattcaaaactgacctcccctggtttagtagggttgatcctgacaagacttattttatccagttttctcaaaaccggagcaacagtagatcttacacaggctgggatcacctgggaaaatataaaacagatgcactaacacttacacaagcagcaattattaatatcggatatcgttttgaggtctttgatgaggccaatgcaactgcaggaatttacacaacaaacaatgctgatttatttgacgaaacaaacgaagcaaaaatgcttccatcggaatatctgtattttttaaaaaactgtgactttgcaggactttacaataaagcattatcagattattggtcaaaaaaccatgaaaagttcaagttattacttaaaaactattatatttcttcatctttatatctttataaaaataacgtaattagcaaagatgaacatgagttcaccatgaaggcattaaatcgggatgataatatagagttgttttcctttgatatatatgggtattactcttctgatattttcggtgctaaaaatgatgacagaataatgcttttcattccgggcgcaaccaatccttttttattctcagaaaatatttctcatttgcggactcatcttaaagaattaataaaagaaaatgataatagagaattactgtcacgtcatttttctctgtatgactgtcaggatggttctactttttatggtgtagattcggttctcaaagaaatagtaaatggaaactttaatgagtcttacttcatgtacacctataaaaaattcaatgagcgggatgtttttgatgctatatcattttccgttcagaaacgtagttttagtgatggcgacaccattattaaatcaaacagtgaagcacaacgggattatgcactgacaataatccaggcaatcgtatcaatgatacctgtgtttgacattatacttccggaagtgtctgttccgcttagcatgggtattatagcctccagtatggggatcagttttgatcagctaattaatggcgatacttatgaagaacgtcgttcggcaattcctggagtagcgacaaatgccgtattgttgggtatatcctttgcactcccgtacctgattagtaaggccagtgaaaataaggtgatacttagtcagactgtaagtaatgaagacagtatattgaatgaaacaaatattgataattttcttgctgaaaatggtattaataaagacgatatacctgcaaatggaatattagaagttgatataaaaaaatccgggatccctgtcaatcttgtaaaaattagtgatgaagacaatcaaattgttgctgtcaggggaagttctcagagtggcatctactatgaagtggatattgaaacagggtatgagattttatcccgaagagtttaccgtacagaatataataatgaaatattctggatacgaaatggcggactgaaagggggacaaccattcgattttgaaaatctcgatatccctacttttttcgtagataaaccttattccgaactggcatcgtctccggaactgtcctttatcaatgatgacagcccactattgtttccttatgtggactcaagattaccgaaaccaacgtcagaaatggatatcagttattactcatctaatttttccagctttgctgagaatactgtaaccctgatgcggggggctactgaagaagaagcatggaatattgcctattataaaacagcaggaggaagcaacaaagaactggaagagatttttataggtggaggaccacaggctaatttaagtttcacggaatacacatcaaacatcagaagtgcagatgcagcaagcagaagacattttcttgtagtcataaacgtaaaaataaaatatatcagcaatgataatgttttatatgctaatcactgggcaattcctgatgaagcccctgttgaagtgctggcggtggtcgaccgtagatttatatttcctgaacctccgaccccaccaaaattatcacttatacaaaaaatatcacaaagattctttacagaagatatagatgaaacatccagaattaattttcagagattaaacagcggtaatattaatgtactaaaaggcagggggagtttatcatcgaaaaatcaacgttcaatttacctgcgttttgatgccgttaatgctgatgatctccgaccagatgagatctacgtcaaaaaagaccagtttgatgacttgggttatgatcggtatttctataataacgctgtgggattagatggctctcccacactgaacacttatactggtgaatttctgacagacccatctttattcggttcactttactggagtaaatataatctgaccaataaaacaagcattattcgtgtagcaaattcagccaggggggcaaacggtatcagaatagcgcttaaagaagttcaggaaaataaacctgtaatcatcacgaatggaaatctaagtggttgtaccacgatcgtcgcacgaaagggagagtatctgtatgaagtgcatactggcacattagagcctctgttaggttttaccagtacaacaggagtaaaaaaagcagtggaagttttgtcgacactcgcagaacaagaaatcccttcactcgcgggaacaataaataatgattttctggtagattttttagcagaaaactttgataaatcattagtaacgtattcatcatcaacactaaaaccagacagcataattaccattagtcgtgataatgtatctacttttccttactatactgacgatattatccatccaggatttggcacatcggtaactatattggtgagaatagatgataatactgttgtaaagtctctgtcagaaagttatgttacaaatgcagatggctccaggatatcagtatttaaggtattatcaaaagattttt

SEQ ID NO: 6 - protein, 1013 aa, Escherichia coli
MNTQWQQKYLLEYNDLVSKFPSPEKVTSDYIKHKFKTDLPWFSRVDPDKTYFIQFSQNRSNSRSYTGWDHLGKYKTDALTLTQAAIINIGYRFEVFDEANATAGIYTTNNADLFDETNEAKMLPSEYLYFLKNCDFAGLYNKALSDYWSKNHEKFKLLLKNYYISSSLYLYKNNVISKDEHEFTMKALNRDDNIELFSFDIYGYYSSDIFGAKNDDRIMLFIPGATNPFLFSENISHLRTHLKELIKENDNRELLSRHFSLYDCQDGSTFYGVDSVLKEIVNGNFNESYFMYTYKKFNERDVFDAISFSVQKRSFSDGDTIIKSNSEAQRDYALTIIQAIVSMIPVFDIILPEVSVPLSMGIIASSMGISFDQLINGDTYEERRSAIPGVATNAVLLGISFALPYLISKASENKVILSQTVSNEDSILNETNIDNFLAENGINKDDIPANGILEVDIKKSGIPVNLVKISDEDNQIVAVRGSSQSGIYYEVDIETGYEILSRRVYRTEYNNEIFWIRNGGLKGGQPFDFENLDIPTFFVDKPYSELASSPELSFINDDSPLLFPYVDSRLPKPTSEMDISYYSSNFSSFAENTVTLMRGATEEEAWNIAYYKTAGGSNKELEEIFIGGGPQANLSFTEYTSNIRSADAASRRHFLVVINVKIKYISNDNVLYANHWAIPDEAPVEVLAVVDRRFIFPEPPTPPKLSLIQKISQRFFTEDIDETSRINFQRLNSGNINVLKGRGSLSSKNQRSIYLRFDAVNADDLRPDEIYVKKDQFDDLGYDRYFYNNAVGLDGSPTLNTYTGEFLTDPSLFGSLYWSKYNLTNKTSIIRVANSARGANGIRIALKEVQENKPVIITNGNLSGCTTIVARKGEYLYEVHTGTLEPLLGFTSTTGVKKAVEVLSTLAEQEIPSLAGTINNDFLVDFLAENFDKSLVTYSSSTLKPDSIITISRDNVSTFPYYTDDIIHPGFGTSVTILVRIDDNTVVKSLSESYVTNADGSRISVFKVLSKDF

SEQ ID NO: 7 - DNA, 3042 bp, Yersinia pseudotuberculosis
  source 1..3042: mol_type = other DNA
atgaaaaatcaatggcaacatcaatattttttatcctattctgagttagttgcaaactttccgtccccagaaaaagtagtaagtgattatattaaacataagtttagtacaacacttccatggtttggctgggcagaccctgacaacctttattttatccgatttactcaaagccgtagtaacaataaatcttataccggatgggatcatcttggtaagtatgccattgaaacgttaaccctcacacaagcagcaatagttaatattggaagtcgttttgatatctttgacgaagctaattcaactgctggaatatataaaacaaacaatgctgattcttttgacgaaacaaacgaagcaaaaatgcttccttccgaatacttatattttttgagagattgtgatttttctaatctctacaataaagcgctatcagattactgggctgagaattacgaaaaatttagcacattactacaaaactattatatttcatcagcgtattatctatataaagatagtgctattagtaaagatgaatatgaattctctattgatgctatttttaataaaaaaagtaaaatattgcgttattactttgatgtttatgggtattattcatctgatatgtttgttgctatgaatgataataaaacaatgctttttattccgggggcaaccaatccctttatcttcgcagacaacattacagatttacgcgacaaaataaaggcgttaatttctgataaaaacacccgtgagttattttctaagcatttttctttatatgaccgacaagatggaaacacgtatttaggtgttaattcaatgctcgaacagattgttagtggtgtggtagatactaattacataatgtattctaataaaaacattcgtgaacgtaacgtatttggatcaatggcgttttctacccgagaaagaagctttaatgatggtgatgtaattattaaatcaaacgcagaagtccagcgagattatgccctgaatgttttacaaaccattttatctttgagccctatatttgatatcgtgctcccggaagtttctattccgatcagccttggtataaccgcttcaagtgtcggtattagttttgatgagttaataaacggggatacttatgaagagcgtcgctctgcaattcctggattggcaacaaatactgtgttattgggtatatcattcgcaataccatttttaatcagtaaggcagaagaaaataagttaattatcaataatttagtaggtagtgacgaaaacatacttaataaaaataatcttggtgattttttggaaaagtacaatattagcgagagtgatattccagaaaatggctctttagttattaatttgaaaaacactaacgtacctgttcgtcttgttaaactgaatgatgaagagggcgaaattgttgcaatcaaaggaagcacattgagcgggatttactacgaagtagatactgaaacaggttatgagatattgtcacgccgggtatttcgaaccgaatataatgaaaaaatttactggacaagggggggaggactgaaaggtggtcagccatttaacttcgaggggcttgatattcctgtttattttatagataaaccatattctgaacttgcatcttcagtggaactttcatttgtgaatgatgatagccctttgttattccctgaaatggactccagattaccaaaaccgactccagagttagatataaaatactactcttctaatttatcttcttttaaagaagatacggtaattctaatgagaggaacgacagaagaagaagcgtggaatattgcaaactataaaacggcaggtgggagcaacaaagatcttgaagagaactttattgaggctggaccacagttcaacttaagtttttctgaatatacatcaagcattaacagtgcagatactgcaagtagaaaacattttcttgtgattatcaaagtacaggtaaaatatattagcaatgataatgttctttatgctaatcactgggccattccagatgaagcacctgtagaagttcttgcagttgtggacagacggtttatcttccctgaaccaccagtaaaaccaaaattatcatttatacagaaaattgccaatagatttctcacagaaaacgtagctgaaatttccagtataaattttcgaagattaaattctggaaacattaatgttctaaaaggaagaggggttttctcatcaagacggttgcgagaaatttatctacgttttgatgcagctaatgctgatgaattacgtccgggagatgtttatgtaaaaaaaacaaagtttgacagtatgggatatgactctcatttctataatgagggaataggcattaatggtgctccaacacttaatacatatacaggggaatatgtcgccgactcatcttctcagggagcaacatactggctcaagtataatctaactaatgaaacaagtatcattaaagtatcaaactctgctcgtggagcaaatggtataaaaattgcgcttgaggaaattgaggaaaataaacctgtcgtaattacctctggtactttaacagggtgtacagttgtttttgcacgaaaaggcgaatacttttatgctgtacatacaggaaattctgagtcacttataggattcaccagtacatcaggtgttgcaaaggcaattgaagtactgtcatctctatctgaacttgaagtccccgcattaccagatgtaataaataacaatacattggttgagtatttatcagataatttcgattcagcattgatttcttattcttcatcatcgctaaaacctaatagcatgattaatattagtagagaaaatgtatctacatttagttattatactgatgatattcaactgccatcttttggtacatccgtaacgatccttgtcaggactaatgataatactgtcgtaaggtctctatcggaaagttatacgatgaattctaatagcagtaagatggttgtgtttaatgttttacaaaaagacttt

SEQ ID NO: 8 - protein, 1014 aa, Yersinia pseudotuberculosis
MKNQWQHQYFLSYSELVANFPSPEKVVSDYIKHKFSTTLPWFGWADPDNLYFIRFTQSRSNNKSYTGWDHLGKYAIETLTLTQAAIVNIGSRFDIFDEANSTAGIYKTNNADSFDETNEAKMLPSEYLYFLRDCDFSNLYNKALSDYWAENYEKFSTLLQNYYISSAYYLYKDSAISKDEYEFSIDAIFNKKSKILRYYFDVYGYYSSDMFVAMNDNKTMLFIPGATNPFIFADNITDLRDKIKALISDKNTRELFSKHFSLYDRQDGNTYLGVNSMLEQIVSGVVDTNYIMYSNKNIRERNVFGSMAFSTRERSFNDGDVIIKSNAEVQRDYALNVLQTILSLSPIFDIVLPEVSIPISLGITASSVGISFDELINGDTYEERRSAIPGLATNTVLLGISFAIPFLISKAEENKLIINNLVGSDENILNKNNLGDFLEKYNISESDIPENGSLVINLKNTNVPVRLVKLNDEEGEIVAIKGSTLSGIYYEVDTETGYEILSRRVFRTEYNEKIYWTRGGGLKGGQPFNFEGLDIPVYFIDKPYSELASSVELSFVNDDSPLLFPEMDSRLPKPTPELDIKYYSSNLSSFKEDTVILMRGTTEEEAWNIANYKTAGGSNKDLEENFIEAGPQFNLSFSEYTSSINSADTASRKHFLVIIKVQVKYISNDNVLYANHWAIPDEAPVEVLAVVDRRFIFPEPPVKPKLSFIQKIANRFLTENVAEISSINFRRLNSGNINVLKGRGVFSSRRLREIYLRFDAANADELRPGDVYVKKTKFDSMGYDSHFYNEGIGINGAPTLNTYTGEYVADSSSQGATYWLKYNLTNETSIIKVSNSARGANGIKIALEEIEENKPVVITSGTLTGCTVVFARKGEYFYAVHTGNSESLIGFTSTSGVAKAIEVLSSLSELEVPALPDVINNNTLVEYLSDNFDSALISYSSSSLKPNSMINISRENVSTFSYYTDDIQLPSFGTSVTILVRTNDNTVVRSLSESYTMNSNSSKMVVFNVLQKDF

SEQ ID NO: 9 - protein, 223 aa, synthetic construct
MGNQWQQKYLLEYNELVSNFPSPERVVSDYIKNCFKTDLPWFSRIDPDNAYFICFSQNRSNSRSYTGWDHLGKYKTEVLTLTQAALINIGYRFDVFDDANSSTGIYKTKSADVFNEENEEKMLPSEYLHFLQKCDFAGVYGKTLSDYWSKYYDKFKLLLKNYYISSALYLYKNGELDEREYNFSMNALNRSDNISLLFFDIYGYYASDIFVAKNNDKVMLFIP

SEQ ID NO: 10 - protein, 465 aa, synthetic construct
GAKKPFLFKKNIADLRLTLKELIKDSDKQQLLSQHFSLYSRQDGVSYAGVNSVLHAIENDGNFNESYFLYSNKTLSNKDVFDAIAISVKKRSFSDGDIVIKSNSEAQRDYALTILQTILSMTPIFDIVVPEVSVPLGLGIITSSMGISFDQLINGDTYEERRSAIPGLATNAVLLGLSFAIPLLISKAGINQEVLSSVINNEGRTLNETNIDIFLKEYGIAEDSISSTNLLDVKLKSSGQHVNIVKLSDEDNQIVAVKGSSLSGIYYEVDIETGYEILSRRIYRTEYNNEILWTRGGGLKGGQPFDFESLNIPVFFKDEPYSAVTGSPLSFINDDSSLLYPDTNPKLPQPTSEMDIVNYVKGSGSFGDRFVTLMRGATEEEAWNIASYHTAGGSTEELHEILLGQGPQSSLGFTEYTSNVNSADAASRRHFLVVIKVHVKYITNNNVSYVNHWAIPDEAPVEVLA

SEQ ID NO: 11 - protein, 47 aa, synthetic construct
VVDRRFNFPEPSTPPDISTIRKLLSLRYFKESIESTSKSNFQKLSRG

SEQ ID NO: 12 - protein, 223 aa, synthetic construct
MNVQWQQKYLLEYNELVSNFPSPERVVSDYIRRCFKTDLPWFSQVDPDNTYFIRFSQSRSNSRSYTGWDHLGKYKTGVLTLTQAALINIGYHFDVFDDANASAGIYKTSSADMFNEKNEEKMLPSEYLYFLKGCDFSGIYGRFLSDYWSKYYDKFKLLLKNYYISSALYLYKNGEIDEYEYNFSISALNRRDNISLFFFDIYGYYSSDMFVAKNNERVMLFIP

SEQ ID NO: 13 - protein, 465 aa, synthetic construct
GAKKPFLFEKNIADLRISLKNLIKENDNKQLLSQHFSLYSRQDGITYAGVNSVLNAIENDGVFNESYFLYSNKRINNKDVFDAVAFSVKKRSFSDGDIVIKSNSEAQRDYALTILQTILSMTPIFDVAIPEVSVTLGLGIIASSMGISFDQLINGDTYEERRSAIPGLATNAALLGLSFAIPFLISKAGTNQKILSRYTKHEIRTLNETNIDMFLEEYGINKNSISETKVLEVELKGSGQHVNIVKLSDEDSKIVAVKGNSLSGIYYEVDIETGYEISSRRIYRTEYNDKIFWTRGGGLKGGQSFDFESLKLPIFFKDEPYSAVPGSSLSFINDDSSLLYPNSTPKLPQPTPEMEIVNYVKRAGDFGERLVTLMRGTTEEEAWNIARYHTAGGSTEELHEILLGQGPQSSLGFTEYTSNINSADAASRRHFLVVIKVQVKYINNNNVSHVNHWAIPDEAPVEVLA

SEQ ID NO: 14 - protein, 47 aa, synthetic construct
VVDRRFNFPEPSTPPNISIIHKLLSLRYFKENIESTSRLNLQKLNRG

SEQ ID NO: 15 - protein, 223 aa, synthetic construct
MNTQWQQKYLLEYNDLVSKFPSPEKVTSDYIKHKFKTDLPWFSRVDPDKTYFIQFSQNRSNSRSYTGWDHLGKYKTDALTLTQAAIINIGYRFEVFDEANATAGIYTTNNADLFDETNEAKMLPSEYLYFLKNCDFAGLYNKALSDYWSKNHEKFKLLLKNYYISSSLYLYKNNVISKDEHEFTMKALNRDDNIELFSFDIYGYYSSDIFGAKNDDRIMLFIP

SEQ ID NO: 16 - protein, 465 aa, synthetic construct
GATNPFLFSENISHLRTHLKELIKENDNRELLSRHFSLYDCQDGSTFYGVDSVLKEIVNGNFNESYFMYTYKKFNERDVFDAISFSVQKRSFSDGDTIIKSNSEAQRDYALTIIQAIVSMIPVFDIILPEVSVPLSMGIIASSMGISFDQLINGDTYEERRSAIPGVATNAVLLGISFALPYLISKASENKVILSQTVSNEDSILNETNIDNFLAENGINKDDIPANGILEVDIKKSGIPVNLVKISDEDNQIVAVRGSSQSGIYYEVDIETGYEILSRRVYRTEYNNEIFWIRNGGLKGGQPFDFENLDIPTFFVDKPYSELASSPELSFINDDSPLLFPYVDSRLPKPTSEMDISYYSSNFSSFAENTVTLMRGATEEEAWNIAYYKTAGGSNKELEEIFIGGGPQANLSFTEYTSNIRSADAASRRHFLVVINVKIKYISNDNVLYANHWAIPDEAPVEVLA

SEQ ID NO: 17 - protein, 46 aa, synthetic construct
VVDRRFIFPEPPTPPKLSLIQKISQRFFTEDIDETSRINFQRLNSG

SEQ ID NO: 18 - protein, 224 aa, synthetic construct
MKNQWQHQYFLSYSELVANFPSPEKVVSDYIKHKFSTTLPWFGWADPDNLYFIRFTQSRSNNKSYTGWDHLGKYAIETLTLTQAAIVNIGSRFDIFDEANSTAGIYKTNNADSFDETNEAKMLPSEYLYFLRDCDFSNLYNKALSDYWAENYEKFSTLLQNYYISSAYYLYKDSAISKDEYEFSIDAIFNKKSKILRYYFDVYGYYSSDMFVAMNDNKTMLFIP

SEQ ID NO: 19 - protein, 465 aa, synthetic construct
GATNPFIFADNITDLRDKIKALISDKNTRELFSKHFSLYDRQDGNTYLGVNSMLEQIVSGVVDTNYIMYSNKNIRERNVFGSMAFSTRERSFNDGDVIIKSNAEVQRDYALNVLQTILSLSPIFDIVLPEVSIPISLGITASSVGISFDELINGDTYEERRSAIPGLATNTVLLGISFAIPFLISKAEENKLIINNLVGSDENILNKNNLGDFLEKYNISESDIPENGSLVINLKNTNVPVRLVKLNDEEGEIVAIKGSTLSGIYYEVDTETGYEILSRRVFRTEYNEKIYWTRGGGLKGGQPFNFEGLDIPVYFIDKPYSELASSVELSFVNDDSPLLFPEMDSRLPKPTPELDIKYYSSNLSSFKEDTVILMRGTTEEEAWNIANYKTAGGSNKDLEENFIEAGPQFNLSFSEYTSSINSADTASRKHFLVIIKVQVKYISNDNVLYANHWAIPDEAPVEVLA

SEQ ID NO: 20 - protein, 742 aa, synthetic construct
MGNQWQQKYLLEYNELVSNFPSPERVVSDYIKNCFKTDLPWFSRIDPDNAYFICFSQNRSNSRSYTGWDHLGKYKTEVLTLTQAALINIGYRFDVFDDANSSTGIYKTKSADVFNEENEEKMLPSEYLHFLQKCDFAGVYGKTLSDYWSKYYDKFKLLLKNYYISSALYLYKNGELDEREYNFSMNALNRSDNISLLFFDIYGYYASDIFVAKNNDKVMLFIPGAKKPFLFKKNIADLRLTLKELIKDSDKQQLLSQHFSLYSRQDGVSYAGVNSVLHAIENDGNFNESYFLYSNKTLSNKDVFDAIAISVKKRSFSDGDIVIKSNSEAQRDYALTILQTILSMTPIFDIVVPEVSVPLGLGIITSSMGISFDQLINGDTYEERRSAIPGLATNAVLLGLSFAIPLLISKAGINQEVLSSVINNEGRTLNETNIDIFLKEYGIAEDSISSTNLLDVKLKSSGQHVNIVKLSDEDNQIVAVKGSSLSGIYYEVDIETGYEILSRRIYRTEYNNEILWTRGGGLKGGQPFDFESLNIPVFFKDEPYSAVTGSPLSFINDDSSLLYPDTNPKLPQPTSEMDIVNYVKGSGSFGDRFVTLMRGATEEEAWNIASYHTAGGSTEELHEILLGQGPQSSLGFTEYTSNVNSADAASRRHFLVVIKVHVKYITNNNVSYVNHWAIPDEAPVEVLAVVDRRFNFPEPSTPPDISTIRKLLSLRYFKESIESTSKSNFQKLSRGNIDVLKG

SEQ ID NO: 21 - DNA, 2220 bp, synthetic construct
  source 1..2220: mol_type = other DNA
ggcaatcagtggcagcagaagtacctgctcgagtacaacgagctggtgtctaacttcccatctcctgagagagtggtgtccgactacatcaagaactgcttcaagaccgatctgccctggttctcccggatcgaccctgacaacgcctacttcatctgcttcagccagaaccggtctaactcccggtcttacaccggctgggatcacctgggcaagtacaagacagaggtgctgacactgacccaggccgctctgatcaacatcggctacagattcgacgtgttcgacgacgccaacagccggaccggcatctacaagaccaagtccgccgatgtgttcaacgaagagaacgaggaaaagatgctgccctccgagtacctgcatctgctgcagaagtgtgactttgccggcgtgtacggcaagaccctgtctgactactggtccaagtactacgacaagttcaagctgctgctgaagaactactacatcagcagcgccctgtacctgtacaagaacggcgagctggatgagcgcgagtacaatttctccatgaacgccctgaaccgctccgacaacatctccctgctgttcttcgacatctacggctactacgcctccgacatcttcgtggccaagaacaacgacaaagtgatgctgttcatccctggcgccaagaagcccttcctgttcaagaagaatatcgccgacctgcggctgaccctgaaagagctgatcaaggacagcgacaacaaacagctgctgtcccagcacttctccctgtactctcgacaggatggcgtgtcctacgctggcgtgaactctgtgctgcacgccatcgagaacgacggcaacttcaacgagtcctacttcctgtactccaacaagaccctcagcaacaaggacgttttcgacgctatcgccatctccgtgaagaagcggtctttctccgacggcgacatcgtgatcaagtccaactctgaggcccagagagactacgccctgaccatcctgcagacaatcctgagcatgacccctatcttcgatatcgtggtgcccgaggtgtcagtgcctctcggactgggcatcatcaccagctctatgggcatctccttcgaccagctgattaacggcgacacctacgaggaaaggcgctctgctattcctggcctggctaccaatgctgtgctgctgggcctgtctttcgctatccctctgctgatctccaaggccggcatcaatcaagaggtcctgtcctccgtgatcaacaacgagggcagaaccctgaacgagacaaacatcgacatctttctgaaagagtacgggatcgccgaggactccatctcctctaccaacctgctggacgtgaagctgaagtcctctggccagcacgtgaacattgtgaagctgtccgacgaggacaaccagatcgtggctgtgaagggctccagcctgtctggcatctactacgaggtggacatcgagacaggctacgagatcctgtccagacggatctaccggaccgagtataacaacgagattctgtggaccagaggcggcggattgaaaggcggccagccattcgatttcgagtccctgaacatccccgtgttcttcaaggacgagccctactctgctgtgaccggctctcctctgtccttcatcaacgacgactctagcctgctgtaccccgacaccaatcctaagctgcctcagcctacctctgagatggatatcgtgaactacgtgaaaggctccggctccttcggcgatagattcgtgacactgatgagaggcgccaccgaggaagaggcctggaatatcgcttcttaccacaccgctggcggctctaccgaggaactgcatgagattcttctcggccagggacctcagtctagcctgggctttaccgagtacacctccaacgtgaactccgccgacgctgcctctagaaggcacttcctggtggtcatcaaggtgcacgtgaagtacatcaccaacaacaacgtgtcctatgtgaaccactgggctatccctgacgaggcccctgtggaagtgttggccgtggtggacagacggttcaacttccctgagccttccacacctcctgacatctccaccatcagaaagctgctgagcctgcggtacttcaaagagagcatcgagtccaccagcaagagcaacttccagaagctgagccggggcaacatcgacgtgctgaaa

SEQ ID NO: 22 - protein, 742 aa, synthetic construct
MNVQWQQKYLLEYNELVSNFPSPERVVSDYIRRCFKTDLPWFSQVDPDNTYFIRFSQSRSNSRSYTGWDHLGKYKTGVLTLTQAALINIGYHFDVFDDANASAGIYKTSSADMFNEKNEEKMLPSEYLYFLKGCDFSGIYGRFLSDYWSKYYDKFKLLLKNYYISSALYLYKNGEIDEYEYNFSISALNRRDNISLFFFDIYGYYSSDMFVAKNNERVMLFIPGAKKPFLFEKNIADLRISLKNLIKENDNKQLLSQHFSLYSRQDGITYAGVNSVLNAIENDGVFNESYFLYSNKRINNKDVFDAVAFSVKKRSFSDGDIVIKSNSEAQRDYALTILQTILSMTPIFDVAIPEVSVTLGLGIIASSMGISFDQLINGDTYEERRSAIPGLATNAALLGLSFAIPFLISKAGTNQKILSRYTKHEIRTLNETNIDMFLEEYGINKNSISETKVLEVELKGSGQHVNIVKLSDEDSKIVAVKGNSLSGIYYEVDIETGYEISSRRIYRTEYNDKIFWTRGGGLKGGQSFDFESLKLPIFFKDEPYSAVPGSSLSFINDDSSLLYPNSTPKLPQPTPEMEIVNYVKRAGDFGERLVTLMRGTTEEEAWNIARYHTAGGSTEELHEILLGQGPQSSLGFTEYTSNINSADAASRRHFLVVIKVQVKYINNNNVSHVNHWAIPDEAPVEVLAVVDRRFNFPEPSTPPNISIIHKLLSLRYFKENIESTSRLNLQKLNRGNIDIFKG

SEQ ID NO: 23 - DNA, 2226 bp, synthetic construct
  source 1..2226: mol_type = other DNA
atgaacgttcaatggcaacaaaaatatcttcttgagtacaatgagttggtatcaaatttcccttcgccagaaagagtagttagcgattacattaggcgttgtttcaaaacagacttaccttggtttagtcaagtggatcctgataacacttattttatacgattttctcaaagtcggagtaatagtagatcttacaccggatgggatcatcttggaaaatataaaacaggagtactgacactcactcaagccgcacttattaatattggctatcattttgatgtttttgatgatgcaaatgcaagtgcgggaatctataaaacaagcagtgcagatatgtttaacgaaaaaaatgaagagaaaatgcttccttcagaatacctttattttttgaaagggtgtgatttttcaggtatttatggtcgatttctgtcagattattggtcgaaatactatgataaatttaagttgttactaaaaaattattatatttcctcagctttatatctttataaaaacggagaaattgatgaatatgaatataatttctctattagcgcattgaatcgtagagataatatatcattattcttctttgatatctatgggtattactcctctgatatgtttgtggccaagaacaatgaacgggtaatgctttttattcctggcgcaaaaaaaccttttttattcgaaaaaaatattgctgatttacgaattagtcttaaaaatctaattaaagagaacgacaataaacagttactatcccagcatttttcgttatatagtcgtcaagatggaattacttatgcgggtgtaaattcagtgctcaatgcaatagaaaatgatggcgtttttaatgaatcttactttctgtactccaataaaaggattaataataaagatgttttcgatgccgtagcattttctgtcaagaaacgtagttttagtgatggtgatatcgttataaaatcaaacagtgaggctcaacgagattatgcactgaccatacttcagactattttatcaatgactcctatatttgatgtcgcaattccggaggtgtctgttacgcttggactggggattattgcttccagtatggggatcagttttgatcagttaattaatggtgatacttatgaagaacgtcgttctgctatccctggtttagccacaaatgcagcattgcttgggttatcttttgcaattccgtttttgattagtaaggcgggcacaaaccagaagatacttagtagatatacaaagcatgagatcaggactctgaatgaaacaaatatcgatatgtttttggaggaatatggaattaataaaaatagtatatcagaaactaaagtattagaagttgagcttaaaggttctgggcagcatgtcaatattgtaaagcttagtgatgaagatagtaaaatcgtcgctgtaaaagggaattctctgagcggcatctactatgaagttgacattgaaacaggatatgaaatttcatccagaagaatttatcgtaccgaatacaatgataaaattttctggactcgaggtggtgggcttaagggggggcaatcgttcgattttgaaagtctcaaactccctatatttttcaaagatgaaccatattctgcagtaccggggtcttcgttatcatttattaatgatgatagctctcttttatatcctaattcgaccccaaaactaccgcaaccaacgccagaaatggagattgttaattacgttaaaagagctggagactttggggagagacttgtaaccttgatgcgaggaacaactgaagaagaagcgtggaatattgcccgttatcatacggcaggaggaagcaccgaagaattgcatgagattttgttggggcaaggcccacagtcgagtttaggttttactgaatatacttcaaatattaacagtgcagatgcggcaagcagacgacattttcttgtagtcataaaagtgcaagtgaaatatataaacaataataacgtttcgcatgttaatcactgggcaattcctgatgaggctccagtagaagtactggctgtggttgacaggagatttaatttccctgagccatcaactccacctaatatatcaattatacacaagttgttatctctgagatattttaaagaaaatatcgaaagtacatcaaggcttaacttacagaaattaaatcgtggtaatattgatatatttaaaggg

SEQ ID NO: 24 - protein, 742 aa, synthetic construct
MNTQWQQKYLLEYNDLVSKFPSPEKVTSDYIKHKFKTDLPWFSRVDPDKTYFIQFSQNRSNSRSYTGWDHLGKYKTDALTLTQAAIINIGYRFEVFDEANATAGIYTTNNADLFDETNEAKMLPSEYLYFLKNCDFAGLYNKALSDYWSKNHEKFKLLLKNYYISSSLYLYKNNVISKDEHEFTMKALNRDDNIELFSFDIYGYYSSDIFGAKNDDRIMLFIPGATNPFLFSENISHLRTHLKELIKENDNRELLSRHFSLYDCQDGSTFYGVDSVLKEIVNGNFNESYFMYTYKKFNERDVFDAISFSVQKRSFSDGDTIIKSNSEAQRDYALTIIQAIVSMIPVFDIILPEVSVPLSMGIIASSMGISFDQLINGDTYEERRSAIPGVATNAVLLGISFALPYLISKASENKVILSQTVSNEDSILNETNIDNFLAENGINKDDIPANGILEVDIKKSGIPVNLVKISDEDNQIVAVRGSSQSGIYYEVDIETGYEILSRRVYRTEYNNEIFWIRNGGLKGGQPFDFENLDIPTFFVDKPYSELASSPELSFINDDSPLLFPYVDSRLPKPTSEMDISYYSSNFSSFAENTVTLMRGATEEEAWNIAYYKTAGGSNKELEEIFIGGGPQANLSFTEYTSNIRSADAASRRHFLVVINVKIKYISNDNVLYANHWAIPDEAPVEVLAVVDRRFIFPEPPTPPKLSLIQKISQRFFTEDIDETSRINFQRLNSGNINVLKGR

SEQ ID NO: 25 - DNA, 2226 bp, synthetic construct
  source 1..2226: mol_type = other DNA
atgaatacccaatggcaacaaaaataccttctggaatataatgatctggtgtcaaaatttccgtcaccagagaaagtaaccagcgactatattaagcataaattcaaaactgacctcccctggtttagtagggttgatcctgacaagacttattttatccagttttctcaaaaccggagcaacagtagatcttacacaggctgggatcacctgggaaaatataaaacagatgcactaacacttacacaagcagcaattattaatatcggatatcgttttgaggtctttgatgaggccaatgcaactgcaggaatttacacaacaaacaatgctgatttatttgacgaaacaaacgaagcaaaaatgcttccatcggaatatctgtattttttaaaaaactgtgactttgcaggactttacaataaagcattatcagattattggtcaaaaaaccatgaaaagttcaagttattacttaaaaactattatatttcttcatctttatatctttataaaaataacgtaattagcaaagatgaacatgagttcaccatgaaggcattaaatcgggatgataatatagagttgttttcctttgatatatatgggtattactcttctgatattttcggtgctaaaaatgatgacagaataatgcttttcattccgggcgcaaccaatccttttttattctcagaaaatatttctcatttgcggactcatcttaaagaattaataaaagaaaatgataatagagaattactgtcacgtcatttttctctgtatgactgtcaggatggttctactttttatggtgtagattcggttctcaaagaaatagtaaatggaaactttaatgagtcttacttcatgtacacctataaaaaattcaatgagcgggatgtttttgatgctatatcattttccgttcagaaacgtagttttagtgatggcgacaccattattaaatcaaacagtgaagcacaacgggattatgcactgacaataatccaggcaatcgtatcaatgatacctgtgtttgacattatacttccggaagtgtctgttccgcttagcatgggtattatagcctccagtatggggatcagttttgatcagctaattaatggcgatacttatgaagaacgtcgttcggcaattcctggagtagcgacaaatgccgtattgttgggtatatcctttgcactcccgtacctgattagtaaggccagtgaaaataaggtgatacttagtcagactgtaagtaatgaagacagtatattgaatgaaacaaatattgataattttcttgctgaaaatggtattaataaagacgatatacctgcaaatggaatattagaagttgatataaaaaaatccgggatccctgtcaatcttgtaaaaattagtgatgaagacaatcaaattgttgctgtcaggggaagttctcagagtggcatctactatgaagtggatattgaaacagggtatgagattttatcccgaagagtttaccgtacagaatataataatgaaatattctggatacgaaatggcggactgaaagggggacaaccattcgattttgaaaatctcgatatccctacttttttcgtagataaaccttattccgaactggcatcgtctccggaactgtcctttatcaatgatgacagcccactattgtttccttatgtggactcaagattaccgaaaccaacgtcagaaatggatatcagttattactcatctaatttttccagctttgctgagaatactgtaaccctgatgcggggggctactgaagaagaagcatggaatattgcctattataaaacagcaggaggaagcaacaaagaactggaagagatttttataggtggaggaccacaggctaatttaagtttcacggaatacacatcaaacatcagaagtgcagatgcagcaagcagaagacattttcttgtagtcataaacgtaaaaataaaatatatcagcaatgataatgttttatatgctaatcactgggcaattcctgatgaagcccctgttgaagtgctggcggtggtcgaccgtagatttatatttcctgaacctccgaccccaccaaaattatcacttatacaaaaaatatcacaaagattctttacagaagatatagatgaaacatccagaattaattttcagagattaaacagcggtaatattaatgtactaaaaggcagg

SEQ ID NO: 26 - protein, 742 aa, synthetic construct
MKNQWQHQYFLSYSELVANFPSPEKVVSDYIKHKFSTTLPWFGWADPDNLYFIRFTQSRSNNKSYTGWDHLGKYAIETLTLTQAAIVNIGSRFDIFDEANSTAGIYKTNNADSFDETNEAKMLPSEYLYFLRDCDFSNLYNKALSDYWAENYEKFSTLLQNYYISSAYYLYKDSAISKDEYEFSIDAIFNKKSKILRYYFDVYGYYSSDMFVAMNDNKTMLFIPGATNPFIFADNITDLRDKIKALISDKNTRELFSKHFSLYDRQDGNTYLGVNSMLEQIVSGVVDTNYIMYSNKNIRERNVFGSMAFSTRERSFNDGDVIIKSNAEVQRDYALNVLQTILSLSPIFDIVLPEVSIPISLGITASSVGISFDELINGDTYEERRSAIPGLATNTVLLGISFAIPFLISKAEENKLIINNLVGSDENILNKNNLGDFLEKYNISESDIPENGSLVINLKNTNVPVRLVKLNDEEGEIVAIKGSTLSGIYYEVDTETGYEILSRRVFRTEYNEKIYWTRGGGLKGGQPFNFEGLDIPVYFIDKPYSELASSVELSFVNDDSPLLFPEMDSRLPKPTPELDIKYYSSNLSSFKEDTVILMRGTTEEEAWNIANYKTAGGSNKDLEENFIEAGPQFNLSFSEYTSSINSADTASRKHFLVIIKVQVKYISNDNVLYANHWAIPDEAPVEVLAVVDRRFIFPEPPVKPKLSFIQKIANRFLTENVAEISSINFRRLNSGNINVLKG

SEQ ID NO: 27 - DNA, 2226 bp, synthetic construct
  source 1..2226: mol_type = other DNA
atgaaaaatcaatggcaacatcaatattttttatcctattctgagttagttgcaaactttccgtccccagaaaaagtagtaagtgattatattaaacataagtttagtacaacacttccatggtttggctgggcagaccctgacaacctttattttatccgatttactcaaagccgtagtaacaataaatcttataccggatgggatcatcttggtaagtatgccattgaaacgttaaccctcacacaagcagcaatagttaatattggaagtcgttttgatatctttgacgaagctaattcaactgctggaatatataaaacaaacaatgctgattcttttgacgaaacaaacgaagcaaaaatgcttccttccgaatacttatattttttgagagattgtgatttttctaatctctacaataaagcgctatcagattactgggctgagaattacgaaaaatttagcacattactacaaaactattatatttcatcagcgtattatctatataaagatagtgctattagtaaagatgaatatgaattctctattgatgctatttttaataaaaaaagtaaaatattgcgttattactttgatgtttatgggtattattcatctgatatgtttgttgctatgaatgataataaaacaatgctttttattccgggggcaaccaatccctttatcttcgcagacaacattacagatttacgcgacaaaataaaggcgttaatttctgataaaaacacccgtgagttattttctaagcatttttctttatatgaccgacaagatggaaacacgtatttaggtgttaattcaatgctcgaacagattgttagtggtgtggtagatactaattacataatgtattctaataaaaacattcgtgaacgtaacgtatttggatcaatggcgttttctacccgagaaagaagctttaatgatggtgatgtaattattaaatcaaacgcagaagtccagcgagattatgccctgaatgttttacaaaccattttatctttgagccctatatttgatatcgtgctcccggaagtttctattccgatcagccttggtataaccgcttcaagtgtcggtattagttttgatgagttaataaacggggatacttatgaagagcgtcgctctgcaattcctggattggcaacaaatactgtgttattgggtatatcattcgcaataccatttttaatcagtaaggcagaagaaaataagttaattatcaataatttagtaggtagtgacgaaaacatacttaataaaaataatcttggtgattttttggaaaagtacaatattagcgagagtgatattccagaaaatggctctttagttattaatttgaaaaacactaacgtacctgttcgtcttgttaaactgaatgatgaagagggcgaaattgttgcaatcaaaggaagcacattgagcgggatttactacgaagtagatactgaaacaggttatgagatattgtcacgccgggtatttcgaaccgaatataatgaaaaaatttactggacaagggggggaggactgaaaggtggtcagccatttaacttcgaggggcttgatattcctgtttattttatagataaaccatattctgaacttgcatcttcagtggaactttcatttgtgaatgatgatagccctttgttattccctgaaatggactccagattaccaaaaccgactccagagttagatataaaatactactcttctaatttatcttcttttaaagaagatacggtaattctaatgagaggaacgacagaagaagaagcgtggaatattgcaaactataaaacggcaggtgggagcaacaaagatcttgaagagaactttattgaggctggaccacagttcaacttaagtttttctgaatatacatcaagcattaacagtgcagatactgcaagtagaaaacattttcttgtgattatcaaagtacaggtaaaatatattagcaatgataatgttctttatgctaatcactgggccattccagatgaagcacctgtagaagttcttgcagttgtggacagacggtttatcttccctgaaccaccagtaaaaccaaaattatcatttatacagaaaattgccaatagatttctcacagaaaacgtagctgaaatttccagtataaattttcgaagattaaattctggaaacattaatgttctaaaagga

SEQ ID NO: 28 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 29 - DNA, 3539 bp, synthetic construct
  source 1..3539: mol_type = other DNA
gtggataaagatgaatcggcattgcggcaacttgtcgacatggcgcttgtaggctacgacggcgtggtggaagagttgctggcgctgccctcggaagagtccggggatcttgcgggtggacgggccaaacgagagaaggccgaattcgcgctgtttggcgaagcgcccaacggggatgaacccatcggccaggatgctcgtacatggttctattatcccaagtaccgcccggtggcggtttccaatttgaagaaaatgcaggcggcgattcgtgctcgtctcgagcctgagtcattgatcctgcagtggttgatcgcactcgatgtctatctcggcgtgttgatcgccgccttgtcccgcactgcgatcagcgacctggtattcgagtatgtcaaggcgcgctacgagatctactacctactgaatcgcgtgccgcatccgctggcggcggcgtacctcaagcgccgccgccagcgtccggtggatcgttcgggacgtctgggctcggtgttcgaacacccgctatggttcgcctacgacgaattggccggcaccgtcgatctggatgccgacatctacgagcaggcgctcgtcgaaagcatcgaacgccgcatggacggagagccggacgacggcagtctggataccgccggacacgacgtatggcgtttgtgccgcgacggcatcaatcggggcgagcaggcgattttccaggcatccggcccatatggcgtggtcgcggatgcagggtatatgcgcaccgttgccgatctggcgtatgccgatgcattggcggattgcctgcatgcccagctgcgcatccgggcgcaaggctcggtcgatagcccgggagacgagatgccacgcaaacttgatgcgtgggaaatcgccaagtttcatctggccgcgacccagcaggcgcgggttgatctgctcgaggcggcgttcgcgctcgactacgccgccttgcgcgatgtgccggtctacggcgattatcgcaatgcgctggctcttcggttcatcaagcgcgaggccttgcggttgctgggggcgcggcgcggcaacgcctctacgatgccggcggttgcggctggcgagtacgacgaaatcgtggccagtggagcagccaatgacgcggcttatgtatccatggccgccgcgttgatcgcgggcgtgctctgcgatctcgagagcgcgcagcgcacgttgcccgtcgtattggccaggtttcggccccttggcgtcttgcgcgattcagaaggctggagcaggaaaccgcgggcatgctgcttggcgaccaggagccggagcctcggggcttcatcagttttaccgattttcgcgatagcgacgcgttcgccagctacgcggagtatgcggcccagttcaacgactatatcgatcaatacagcatactcgaggcgcagcggttggcgcggattctggccctgggctcgcggatgacggtcgatcaatggtgccttcccctgcagaaagtacggcactacaaggtgctgacatcgcagccagggctgatcgcgcgtggaatcgaaaatcacaacaggggcattgaatattgcctggggcggccgccgctgaccgatctgccgggtcttttcaccatgttccagctccatgattccagctggctgttggtatcgaacatcaacggtgagctttggtctgatgtccttgcgaacgctgaggtgatgcagaatcctacgctggccgcccttgccgagccgcaaggccgctttcgaaccggccgtcgaacgggcgggtggtttctcggcggccccgcgactgaagggccaagtctgcgcgacaactacctcctcaaattgaggcagagcaatccggggctggatgtcaagaaatgctggtatttcgggtatcggcaagagtacaggctgcccgcaggcgcgctgggtgtgccgctattcgctgtttccgttgccctgaggcacagtctggacgacctcgcggcgcacgcgaagtccgctttgtacaaacccagcgaatggcagaagttcgccttctggatcgtgccgttctaccgggaaatattcttttcgacccaggatcgttcctatcgggtcgatgtggggagcatcgtctttgattcgatttccttgcttgcctcggtgttcagcataggagggaagttgggtagtttcacccgtacccagtatggcaacctgcgcaatttcgtggtgcggcaacgcattgcggggttgagcgggcagcgcttgtggcgttcggtgctcaaggagcttccggcattgatcggagccagcgggttgcgcctgtcgcgttcgctgctcgtcgatctgtatgagatcttcgagcccgtgccgatccgtcggcttgtcgcgggattcgtgagcaccactacggtcggcgggcgtaaccaggccttcctgcggcaggcattctccgctgccagttcctcggccgggcgcacggggggccaattggcaagcgaatggcggatggccggcgtggacgccaccggtttggtcgagtccacgtccggcggcaggtttgagggcatctacacgcgcggcttgggaccgttgagcgagcgcaccgagtacttcatcgtcgaatccgggaatgcctacagagtcatctgggatgcatacacgcatggttggcgcgtggtcaacgggcgcttgccgccgaggctgacctataccgttccggttcggctgaacgggcaaggccactgggagacgcatctggatgtccctggccggggtggggcgccggaaatcttcggacgcatccgcacgcgtaatctggtcgcccttgccgccgagcaggccgcgccgatgcgccgcctgctcaaccaggcgaggcgtgtggcgctcaggcatatcgatacctgcaggagcaggcttgcgtcgccgcgcgctgaatccgatatggacgcggcgatccggattttcttcggcgagccggacgccggccttcgccagcgcatcgggcgacgcctgcaggaggtcagggcctatatcggcgatctgagtccggtcaatgacgtgctgtaccgggcgggatatgacctcgacgatgtcgcaacgctgtttaacgcagtggaccggaacacgtcgctgggcaggcaggctcggatggagttgtatctggacgccattgtcgatttgcatgccaggctcggctatgaaaatgcgcgttttgtcgacctgatggcgttccacctgctcagcctgggccatgccgcgacggccagtgaggtcgtggaggccgtttcgccccggctgctgggcaatgtgttcgatatctcgaacgtcgcccagctcgaacgcggtatcgggaatcccgccagcaccggtcttttcgtcatgctgggcgcctattcggaatcgtcgcccgcgatattccagtcgttcgtgaacgatatatttcccgcatggcgacaggcttccggcggggggccgctggtatggaacttcggtcctgccgccatcagcccgacgcgcctggattacgcaaataccgatatcggattgctcaac

SEQ ID NO: 30 - DNA, 41 bp, synthetic construct
  source 1..41: mol_type = other DNA
acgacaagcttgcggccgcgaattcacatcaccatcaccat

SEQ ID NO: 31 - DNA, 41 bp, synthetic construct
  source 1..41: mol_type = other DNA
gatgccacccgggatcctctccaggccgatgctgtacttct

SEQ ID NO: 32 - DNA, 40 bp, synthetic construct
  source 1..40: mol_type = other DNA
caaatggctctagaggtacccgttacataacttacggtaa

SEQ ID NO: 33 - DNA, 21 bp, synthetic construct
  source 1..21: mol_type = other DNA
ccgatgctgtacttcttgtcg

SEQ ID NO: 34 - DNA, 21 bp, synthetic construct
  source 1..21: mol_type = other DNA
gacaagaagtacagcatcggc

SEQ ID NO: 35 - DNA, 20 bp, synthetic construct
  source 1..20: mol_type = other DNA
ggtacctctagagccatttg

SEQ ID NO: 36 - DNA, 20 bp, synthetic construct
  source 1..20: mol_type = other DNA
gggcgaggagctgttcaccg

SEQ ID NO: 37 - DNA, 24 bp, synthetic construct
  source 1..24: mol_type = other DNA
aaaccggtgaacagctcctcgccc

SEQ ID NO: 38 - DNA, 36 bp, synthetic construct
  source 1..36: mol_type = other DNA
cttgtcgtcatcgtctttgtagtcgtcaacttcgtc

SEQ ID NO: 39 - DNA, 30 bp, synthetic construct
  source 1..30: mol_type = other DNA
gccgcgaattcacatcaccatcaccatcac

SEQ ID NO: 40 - DNA, 36 bp, synthetic construct
  source 1..36: mol_type = other DNA
gtgatggtgatggtgatgtgaattcgcggccgcaag

SEQ ID NO: 41 - DNA, 40 bp, synthetic construct
  source 1..40: mol_type = other DNA
acccaggttaccgttagcagcagaggatcccgggtggcat

SEQ ID NO: 42 - DNA, 39 bp, synthetic construct
  source 1..39: mol_type = other DNA
gactacaaagacgatgacgacaaggttcagctggttgaa

SEQ ID NO: 43 - DNA, 35 bp, synthetic construct
  source 1..35: mol_type = other DNA
gatgccacccgggatcctctgctgctaacggtaac

SEQ ID NO: 44 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 45 - DNA, 4101 bp, synthetic construct
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 46 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 47 - DNA, 3684 bp, synthetic construct
  source 1..3684: mol_type = other DNA
atgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcac

SEQ ID NO: 48 - DNA, 23 bp, synthetic construct
  source 1..23: mol_type = other DNA
gattaaacccggccactttcagg

SEQ ID NO: 49 - DNA, 23 bp, synthetic construct
  source 1..23: mol_type = other DNA
cctgaaagtggccgggtttaatc

SEQ ID NO: 50 - DNA, 20 bp, synthetic construct
  source 1..20: mol_type = other DNA
tgtgctagacatgaggtcta

SEQ ID NO: 51 - DNA, 20 bp, synthetic construct
  source 1..20: mol_type = other DNA
tagacctcatgtctagcaca

SEQ ID NO: 52 - DNA, 23 bp, synthetic construct
  source 1..23: mol_type = other DNA
agggagtcctctatgtaggtagg

SEQ ID NO: 53 - DNA, 23 bp, synthetic construct
  source 1..23: mol_type = other DNA
cctacctacatagaggactccct

(19) United States
(12) Patent Application Publication
(10) Pub. No.: US 2026/0137792 A1
(43) Pub. Date: May 21, 2026

(54) CYTOTOXIC NECROTIZING FACTORS AS EXOGENOUS CARRIERS IN MAMMALIAN CELLS

(71) Applicant: SE THERAPEUTICS, Evry-Courcouronnes (FR)

(72) Inventor: Daniel HAMAOUI, PARIS (FR)

(73) Assignee: SE THERAPEUTICS, Evry-Courcouronnes (FR)

(21) Appl. No.: 19/119,842

(22) PCT Filed: Sep. 19, 2023
(86) PCT No.: PCT/EP2023/075850

§ 371 Date: Apr. 10, 2025

(30) Foreign Application Priority Data
Oct. 13, 2022 (EP) 22306551.7

(51) Int. Cl. A61K 47/64; A61K 48/00; C07K 14/245; C12N 15/11; C12N 15/90

(52) U.S. Cl. CPC A61K 47/6415; A61K 48/0033; C07K 14/245; C12N 15/11; C12N 15/907; C12N 2310/20

(57) ABSTRACT

A cell-penetrating polypeptide (CPP), which includes an amino acid sequence that is derived from, or is, a portion of a virulence factor, as well as a complex including the CPP, a polynucleotide encoding the CPP or complex, an expression vector encoding the CPP or complex, and a cell including the same. Also, methods for directing or delivering a molecule of interest into cells using the CPP, and to the use of the CPP for preventing or treating a disease.

### FIELD OF INVENTION

The present invention relates a cell-penetrating polypeptide (CPP), which comprises an amino acid sequence that is derived from, or consists of, a portion of a virulence factor, as well as a complex comprising said CPP, a polynucleotide encoding said CPP or complex, an expression vector encoding said CPP or complex, and a cell comprising the same. The present invention also relates to methods for directing or delivering a molecule of interest into cells using said CPP, and to said CPP for use as a medicament, for preventing or treating a disease.

### BACKGROUND OF INVENTION

Pathogenic bacteria can invade host cells through different mechanisms. Several pathogens utilize virulence factors like toxins to enter targeted cells as, for example, uropathogenic Escherichia coli use cytotoxic necrotizing factor 1 (CNF1). In addition to CNF1, E. coli strains also express homologues of CNF1 such as cytotoxic necrotizing factor 2 (CNF2) and cytotoxic necrotizing factor 3 (CNF3). CNF1, CNF2 and CNF3 are members of the CNF family gathering numerous CNF factors from diverse bacteria like Yersinia, Salmonella or Bordella species. These bacterial virulence factors display a Rho GTPase activator domain that enables the control of invaded cell actin cytoskeleton dynamics, promoting thereby bacterial invasion and infection.

The CNF enter targeted mammalian cells through endocytosis after binding to plasma membrane receptors. Following the acidification of the endosomal compartment a conformational change of the CNFs occurs, leading to the translocation of the catalytic domain (Rho GTPase activator domain) across the membrane of the endosome, thereby reaching the cytosol of the cell.

The intracellular delivery of proteins or other molecules in vitro is performed using electroporation or chemical products that permeabilize the plasma membrane. For therapeutics use, crossing the plasma membrane remains a hurdle to overcome. More often, the targeting of a drug or peptide using an antibody or receptor binding protein to a specific receptor, leads to the endocytosis of the drug followed by endosomal entrapment, extracellular recycling, or targeting to very acidic compartments such as lysosomal vesicles for destruction. Thus, there is still a need for simple carriers that can easily deliver conjugated cargos into the cytoplasm, avoiding cargo modification or destruction in the endosomal pathway.

The present invention consists of using the natural ability of some bacterial virulence factors, such as e.g. Cytotoxic Necrotizing factors (CNFs), to escape endosomal vesicles and release their catalytic domain into the cytoplasm to deliver molecules.

The applicant was surprisingly able to deliver intact and functional molecules of interest inside mammalian cells by replacing the catalytic domain of virulence factors with molecules of interest.

Therefore, the invention relates to the use of virulence factors as carriers for drug delivery to targeted cells.

### SUMMARY

One aspect to the invention relates to a cell-penetrating polypeptide (CPP), which comprises an amino acid sequence that is derived from, or consists of, a portion of a virulence factor, wherein said CPP is associated, or suitable to be associated, to a heterologous cargo.

In some embodiments, said virulence factor is a bacterial virulence factor. In some embodiments, said bacterial virulence factor is derived from, or consists of, a Rho GTPase activator. In some embodiments, said Rho GTPase activator is a Cytotoxic Necrotizing Factor (CNF) family member.

In some embodiments, the CNF member is chosen among Cytotoxic Necrotizing Factor 1 (CNF1), Cytotoxic Necrotizing Factor 2 (CNF2), Cytotoxic Necrotizing Factor 3 (CNF3), and Cytotoxic Necrotizing Factor γ (CNFγ).

In some embodiments, the CPP comprises from N-terminus to C-terminus:
a first receptor binding domain of a virulence factor, and
one translocation domain of a virulence factor.

In some embodiments, the CPP does not comprise the catalytic domain of the virulence factor from which the CPP is derived. In some embodiments, the CPP does not comprise the catalytic domain of any virulence factor.

In some embodiments, the first receptor binding domain of the CPP is a Laminin Receptor binding domain.

In some embodiments, the Laminin Receptor binding domain comprises or consists of an amino acid sequence that is at least 85% identical to SEQ ID NO: 9, SEQ ID NO: 12, SEQ ID NO: 15, or SEQ ID NO: 18.

In some embodiments, the translocation domain of the CPP is an acidic endosome to cytosol translocation domain.

In some embodiments, the translocation domain of the cell-penetrating polypeptide comprises or consists of an amino acid sequence that is at least 85% identical to SEQ ID NO: 10, SEQ ID NO: 13, SEQ ID NO: 16, or SEQ ID NO: 19.

In some embodiments, the CPP further comprises a second receptor binding domain.

In some embodiments, the second receptor binding domain of the CPP comprises or consists of an amino acid sequence that is at least 85% identical to SEQ ID NO: 11, SEQ ID NO: 14, or SEQ ID NO: 17.

In some embodiments, the CPP comprises or consists of an amino acid sequence that is at least 85% identical to SEQ ID NO: 20, SEQ ID NO: 22, SEQ ID NO: 24, or SEQ ID NO: 26.

Another aspect of the invention concerns a complex comprising the cell-penetrating polypeptide (CPP) according to the invention.

In some embodiments, the complex comprises the cell-penetrating polypeptide (CPP) according to the invention associated, e.g. conjugated, to a heterologous cargo.

In some embodiments, the cargo associated or conjugated to the complex is linked to the C-terminus of the CPP.

In some embodiments, the cargo is a nucleic acid molecule, an amino acid molecule, a therapeutically active peptide, a protein, an antibody, a ribonucleoprotein, an enzyme, a transcription factor, a carbohydrate, a lipid, a glycan, a contrast or imaging agent, a quantum dot, a diagnostic agent, a therapeutic agent, and any combination thereof.

In one aspect, the invention relates to a polynucleotide or a set of polynucleotides encoding the cell-penetrating polypeptide (CPP) according to the invention, or encoding the cell-penetrating polypeptide (CPP) of the invention and encoding and/or comprising the heterologous cargo or a portion of the heterologous cargo.

In another aspect, the invention concerns a recombinant expression vector comprising the polynucleotide or set of polynucleotides of the invention.

Another aspect of the invention is a cell comprising a cell-penetrating polypeptide (CPP) according to the invention, a complex according to the invention, a polynucleotide or set of polynucleotides according to the invention, or a recombinant expression vector according to the invention.

In another aspect, the invention relates to an in vitro method for directing or delivering a molecule of interest into a cell comprising contacting the cell with the complex according to the invention, with a polynucleotide or set of polynucleotides according to the invention, or with the recombinant expression vector according to the invention.

In some embodiments, the cell comprising a molecule of interest, a cell-penetrating polypeptide (CPP) according to the invention, a complex according to the invention, a polynucleotide according to the invention, or a recombinant expression vector according to the invention has been targeted through the method are mammalian cells.

Another aspect of the invention is a method for delivering at least one molecule of interest into a cell comprising contacting the cell with the complex according to the invention, a polynucleotide or set of polynucleotides according to the invention or with the recombinant expression vector according to the invention.

Another aspect of the invention is a cell-penetrating polypeptide (CPP) according to the invention, a complex according to the invention, a polynucleotide or set of polynucleotides according to the invention, or a recombinant expression vector according to the invention, for use as a medicament, in particular, for use in the prevention and/or treatment of a disease.

Another aspect of the invention is a method for preventing and/or treating a disease in a subject in need thereof, comprises administering to the subject a cell-penetrating polypeptide (CPP) according to the invention, a complex according to the invention, a polynucleotide or set of polynucleotides according to the invention, or a recombinant expression vector according to the invention.

### Definitions

In the present invention, the following terms have the following meanings:

“Acidic endosome” refers to an endosome vesicle with a lower pH than the regular pH of the cytoplasm (e.g. pH=6.5 instead of pH=6.8 in the cytoplasm).

“Amino acid” is understood to include the 20 naturally occurring amino acids; those amino acids are often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, in some embodiments, the term “amino acid” includes both D- and L-amino acids (stereoisomers). “Amino acid” refers to both natural and synthetic amino acids, and both D- and L-amino acids. They are represented by their full name, their three-letter code or their one-letter code as well-known in the art. Amino acid residues in peptides are thus abbreviated as follows: phenylalanine is Phe or F; leucine is Leu or L; isoleucine is Ile or I; methionine is Met or M; valine is Val or V; serine is Ser or S; proline is Pro or P; threonine is Thr or T; alanine is Ala or A; tyrosine is Tyr or Y; histidine is His or H; glutamine is Gln or Q; asparagine is Asn or N; lysine is Lys or K; aspartic acid is Asp or D; glutamic acid is Glu or E; cysteine is Cys or C; tryptophan is Trp or W; arginine is Arg or R; and glycine is Gly or G. “Standard amino acid” or “naturally occurring amino acid” means any of the twenty standard L-amino acids commonly found in naturally occurring peptides. “Non-standard amino acid” means any amino acid, other than the standard amino acids, regardless of whether it is prepared synthetically or derived from a natural source. For example, naphtlylalanine can be substituted for tryptophan to facilitate synthesis. Other synthetic amino acids that can be substituted include, but are not limited to, L-hydroxypropyl, L-3,4-dihydroxyphenylalanyl, alpha-amino acids such as L-alpha-hydroxylysyl and D-alpha-methylalanyl, L-alpha-methylalanyl, beta-amino acids, and isoquinolyl. The CPP fragments of the invention may comprise standard amino acids or non-standard amino acids. The term “amino acid” also encompasses chemically modified amino acids, including, but not limited to, salts, amino acid derivatives (such as amides), and substitutions.

“Antibody” refers to a protein having a combination of two heavy and two light chains which have significant known specific immunoreactive activity to an antigen of interest (e.g. EGFP, TSP1, HTRA1 or Fas). Antibodies and immunoglobulins comprise light and heavy chains, with or without an interchain covalent linkage between them. Basic immunoglobulin structures in vertebrate systems are relatively well understood. The generic term “immunoglobulin” comprises five distinct classes of antibody that can be distinguished biochemically. Although the following disclosure will generally be directed to the IgG class of immunoglobulin molecules, all five classes of antibodies are within the scope of the present invention. With regard to IgG, immunoglobulins comprise two identical light polypeptide chains of molecular weight of about 23 kDa, and two identical heavy chains of molecular weight of about 53-70 kDa. The four chains are joined by disulfide bonds in a “Y” configuration wherein the light chains bracket the heavy chains starting at the mouth of the “Y” and continuing through the variable region. The light chains of an antibody are classified as either kappa (κ) or lambda (λ). Each heavy chain class may be bonded with either a κ or λ light chain. In general, the light and heavy chains are covalently bonded to each other, and the “tail” regions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma (γ), mu (μ), alpha (α), delta (δ) or epsilon (ε) with some subclasses among them (e.g., γ1-γ4). It is the nature of this chain that determines the “class” of the antibody as IgG, IgM, IgA IgD or IgE, respectively. The immunoglobulin subclasses or “isotypes” (e.g., IgG1, IgG2, IgG3, IgG4, IgA1, etc.) are well characterized and are known to confer functional specialization. Modified versions of each of these classes and isotypes are readily discernable to the skilled artisan in view of the instant disclosure. As indicated above, the variable region of an antibody allows the antibody to selectively recognize and specifically bind epitopes on antigens. That is, the light chain variable domain (V_{L} domain) and heavy chain variable domain (V_{H} domain) of an antibody combine to form the variable region that defines a three-dimensional antigen binding site. This quaternary antibody structure forms the antigen binding site presents at the end of each arm of the “Y”. More specifically, the antigen binding site is defined by three complementarity determining regions (CDRs) on each of the V_{H} and V_{L} chains.

“Antibody fragment”, as used herein, refers to a part or region of an antibody which comprises fewer amino acid residues than the whole antibody. An “antibody fragment” binds antigen and/or competes with the whole antibody from which it derives for antigen binding. Antibody fragments encompasses, without any limitation, a single chain antibody, a dimeric single chain antibody, a Fv, a scFv, a Fab, a Fab′, a Fab′-SH, a F(ab)′2, a Fd, a defucosylated antibody, a diabody, a triabody and a tetrabody. It may also encompass a unibody, a domain antibody, and a nanobody.

“CMV promoter” refers to the nucleic acids sequence that enable the control of Cytomegaloviruses coding sequence transcription. An example of a nucleic acid sequence of CMV promotor is SEQ ID NO: 29.

“Cargo” refers to a molecule associated, for instance conjugated, to the cell penetrating polypeptide, which is, or is to be, delivered to a cell. Non-limiting examples of said molecules are nucleic acids (e. g. siRNA, sgRNA, mRNA, tRNA, miRNA, cDNA, DNA etc.), amino acids, therapeutically active peptides, proteins, peptides, polypeptides, antibodies, ribonucleoproteins, enzymes, transcription factors, carbohydrates, lipids, glycans, contrast or imaging agents, quantum dots, diagnostic agents, therapeutic agents, and any combination thereof.

“Carrier” refers to a polypeptide that has the ability to be associated to, or bind to, and to transport a cargo as defined hereinabove.

“Cell-penetrating polypeptide” refers to a polypeptide able to cross the cellular plasma membrane by itself due to specific domains (e.g. ligand to plasma membrane component, hydrophobic transmembrane domains).

“CNF family member” refers to any Cytotoxic Necrotizing Factor including Cytotoxic Necrotizing Factor 1 (CNF1), Cytotoxic Necrotizing Factor 2 (CNF2), Cytotoxic Necrotizing Factor 3 (CNF3), Cytotoxic Necrotizing Factor γ (CNFγ).

“Complex” or “complex of molecules” refers to molecules of the same or different type, as defined above, associated or linked together. For instance, the association between the molecules is made by complementary base matching, covalent bond, ionic bond, hydrogen bond, polar bond, hydrophobic interaction or effect (van der Waals force), electrostatic interaction or forces (e.g. between a RNA or DNA and a protein), biotin/streptavidin interaction, and the like.

“Associated to” or “linked to” refers to any type of link possible between molecules as defined previously. This association or link may be made via a linker herein defined or not, and may be made by complementary base matching, covalent bond, ionic bond, hydrogen bond, polar bond, hydrophobic interaction or effect (van der Waals force), electrostatic interaction or forces (e.g. between a RNA or DNA and a protein), biotin/streptavidin interaction, and the like. In some embodiments, the associated molecules are conjugated by a covalent bond.

“CRISPR associated protein” refers to proteins with the ability to break hydrogen bonds of double stranded DNA when in complex with single guide (sg)RNA according to the Genome editing technique (The new frontier of genome engineering with CRISPR-Cas9; Jennifer A Doudna, Emmanuelle Charpentier, Science 2014). “CRISPR associated proteins” are enzymes that associate with CRISPR RNAs to bind to and alter DNA or RNA target sequences. Cas enzymes originate in bacteria and, as such, a wide variety of types exist. Some are used for genome editing or RNA editing. Some versions have been altered by humans to perform specific tasks. Common examples of CRISPR associated proteins include Cas9 which makes a double-stranded break in a target DNA sequence, and Cas13 which targets RNA.

“Expression vector” refers to an engineered construction allowing the transcription and/or the translation of a DNA (or cDNA) or RNA sequence in a host cell. Expression vectors may comprise a nucleic acid sequence encoding a polypeptide according to the invention operatively associated with expression control elements. This construction may be a plasmid, cosmid, fosmid, episome, artificial chromosome, phage, viral vector and any vector able to be transcribed or translated in eukaryotic cells. The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. The expression vector according to the invention may comprise a functional expression cassette. An expression cassette comprises a nucleic acid sequence encoding a polypeptide of the invention, which is operably linked to elements necessary to its expression. Said vector advantageously contains a promoter sequence, signals for initiation and termination of translation, as well as appropriate regions for regulation of translation, such as a promoter, enhancer, terminator and the like, to cause or direct expression of said polypeptide upon administration to a subject. Examples of promoters and enhancers used in the expression vector for animal cell include early promoter and enhancer of SV40, LTR promoter and enhancer of Moloney mouse leukemia virus, promoter and enhancer of immunoglobulin H chain. Insertion of said vector into the host cell may be transient or stable. The vector may also contain sequences encoding specific signals which trigger the secretion of the translated protein or its targeting to cellular compartments or organelles (e.g; Golgi apparatus, endosomes, periplasm . . . ). These various control signals are selected according to the host cell and may be inserted into vectors which self-replicate in the host cell, or into vectors which integrate the genome of said host.

Any expression vector for animal cell can be used. Examples of suitable vectors include e.g. pAGE107, pAGE103, pHSG274, pKCR, pSG1 beta d2-4-. Other examples of plasmids include replicating plasmids comprising an origin of replication, or integrative plasmids, such as for instance pUC, pcDNA, pBR. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc.

“Fusion protein” or “fusion polypeptide” interchangeably refer to a synthetic association of at least two proteins or peptides or polypeptides. In some embodiments, this association is made by translation of a designed mRNA sequence.

The term “identity” refers to a measure of the identity of nucleotide sequences or amino acid sequences. In general, the sequences are aligned so that the highest order match is obtained. “Identity” per se has an art-recognized meaning and can be calculated using published techniques. Methods to determine identity and similarity are codified in computer programs. Computer program methods to determine identity and similarity between two sequences include, but are not limited to, GCG program package, the GAP program. As an illustration, by a polynucleotide having a nucleotide sequence having at least, for example, 95% “identity” to a reference nucleotide sequence is intended that the nucleotide sequence of the polynucleotide is identical to the reference sequence except that the polynucleotide sequence may include an average up to five point-mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

“Laminin receptor binding domain” refers to an amino acidic domain possessing the ability to specifically bind Laminin Receptors. Laminins are a family of glycoproteins of the extracellular matrix of all animals. They are major components of the basal lamina (one of the layers of the basement membrane), the protein network foundation for most cells and organs. Laminins are an important and biologically active part of the basal lamina, influencing cell differentiation, migration, and cell adhesion. Laminins are recognized by receptors that are anchored to the plasma membrane, thereby mediating signals from the extracellular matrix to the cytoskeleton of the cells.

“Linker”, also called “peptide linker” or “spacer linker”, interchangeably refers to an amino acid sequence (generally of short length), typically a synthetic amino acid sequence, that connects or links a peptide or a polypeptide with another peptide or polypeptide or with another molecule. Linkers typically connect two peptide or polypeptide sequences via peptide bonds. Linkers are well-known in the art. Examples of suitable linkers include so-called “GS linkers” or “Gly-Ser linkers”, i.e., amino acid sequences essentially consisting of glycine (G) and serine (S) residues, and usually—but not always—comprising two or more repeats of a peptide motif. GS linkers are well-know and widely used in the art, in particular for their flexibility properties.

“Mammalian cells” refers to cells that derive from any mammal, including humans, domestic, lab and farm animals, and zoo, sports, or pet animals, such as dogs, mice, primates, cattle, horses, sheep, pigs, goats, rabbits, etc. Preferably, the mammal is a human.

“Nanobody” refers to a single-domain antibody which is an antibody fragment consisting of a single monomeric variable antibody domain engineered from camelids heavy-chain-only antibodies. In some embodiment, the single domain antibody targets an exogenous or endogenous protein expressed in eukaryotic cells. In other embodiments, the single domain antibody is directed against a pathogenic antigen.

“Nucleic acid” refers to any layout of naturally occurring nucleic acid. In some embodiments nucleic acids are deoxyribonucleic acids (DNA) composed of A (adenine), T (thymine), C (cytosine), G (guanine) bases. In other embodiments, nucleic acids are ribonucleic acids composed of A (adenine), U (uracil), C (cytosine), G (guanine) bases.

“Optimized sequence” refers to DNA sequence bearing codon modifications to favor transcription of the mRNA in a specific species. In some embodiments, the codons are optimized to promote transcription of the DNA sequence of a CNF family member in mammalian cells.

“Peptide” refers to a linear polymer of amino acids of less than 50 amino acids linked together by peptide bonds.

“Polypeptide” refers to a linear polymer of amino acids of more than 50 amino acids linked together by peptide bonds.

“Receptor binding domain” refers to an amino acid domain that specifically possesses the ability to bind or be transiently or definitively linked to a cellular receptor.

“Rho GTPase activator” refers to any protein that has the ability to deamidate glutamine to glutamate in the active site (called switch II) of the Rho family of proteins (including RhoA, Rac1 and Cdc42) resulting in their constitutive activation.

“Single chain antibody” or “scFv” refers to antibody fragments that comprise the V_{H} and V_{L} antibody domains connected into a single amino acid chain. Preferably, the scFv amino acid sequence further comprises a peptide linker between the V_{H} and V_{L} domains that enables the scFv to form the desired structure for antigen binding. “Fv” refers to the minimum antibody fragment that contains a complete antigen recognition and binding site. This fragment consists of a dimer of one heavy-chain variable region and one light-chain variable region in tight non-covalent association.

“Single-domain antibodies” refer to antibody-derived proteins that contain the unique structural and functional properties of naturally-occurring heavy chain-only antibodies. These heavy chain antibodies may contain a single variable domain (V_{H}H)—one such example is Nanobodies®—or a single variable domain (V_{H}H) and two constant domains (C_{H}2 and C_{H}3)—such as camelid antibodies—or a single variable domain (V_{H}H) and five constant domains (C_{H}1, C_{H}2, C_{H}3, C_{H}4 and C_{H}5)—such as shark antibodies.

“Subject” refers to a mammal, preferably a human. In some embodiments, the subject is a “patient”, i.e., a mammal, preferably a human, who/which is awaiting the receipt of, or is receiving medical care or was/is/will be the object of a medical procedure, or is monitored for the development of a disease. In some embodiments, the subject is an adult (for example a subject above the age of 18). In other embodiments, the subject is a child (for example a subject below the age of 18). In some embodiments, the subject is a male. In other embodiments, the subject is a female.

A “therapeutic protein” as used herein is typically a peptide, a polypeptide or a protein, which is beneficial for the treatment or prophylaxis of any inherited or acquired disease or which improves the condition of a subject. In particular, therapeutic proteins may play a role in the modification and repair of genetic deficiencies, in the destruction of cancer cells or of pathogens and pathogen infected cells, and/or in the treatment or prevention of various diseases including immune system disorders such as auto immune diseases, metabolic or endocrine disorders, diseases of the nervous system, diseases of the circulatory system, diseases of the respiratory system, diseases of the digestive system, diseases of the skin and subcutaneous tissue, diseases of the musculoskeletal system and connective tissue, diseases of the genitourinary system, etc., independently if they are inherited or acquired. In the context of this invention, the term “therapeutic protein” typically refers to both peptides and proteins. It may also refer to a polypeptide or polyprotein comprising a therapeutic protein as defined herein. For instance, the term may refer to a polypeptide comprising the therapeutic protein fused, preferably in N-terminal or C-terminal, to a further amino acid sequence which is not derived from the therapeutic protein.

“Therapeutic molecule” refers to any molecule (peptide, polypeptide, enzyme, RNA, DNA, lipid, etc.) that may have a benefic effect on cell functioning or organization is beneficial for the treatment or prophylaxis of any inherited or acquired disease or which improves the condition of a subject.

“Therapeutically effective amount” means the level or amount of an agent that is aimed at, without causing significant negative or adverse side effects to the target, (1) delaying or preventing the onset of a disease; (2) slowing down or stopping the progression, aggravation, or deterioration of one or more symptoms of a disease; (3) bringing about ameliorations of the symptoms of a disease; (4) reducing the severity or incidence of a disease; or (5) curing a disease. A therapeutically effective amount may be administered prior to the onset of the disease, for a prophylactic or preventive action. Alternatively, or additionally, the therapeutically effective amount may be administered after initiation of the disease, for a therapeutic action or maintenance of a therapeutic action.

“Treating” or “treatment” or “alleviation” refers to both therapeutic treatment and prophylactic or preventative measures; wherein the object is to prevent or slow down (lessen) the targeted disease or pathologic condition or disorder. Those in need of treatment include those already with the disorder as well as those prone to have the disorder or those in whom the disorder is to be prevented. A subject, patient or mammal is successfully “treated” for the targeted pathologic disorder if, after receiving a therapeutic amount of the composition of the present invention, the patient shows observable effects on one or more of the followings; relief to some extent, of one or more of the symptoms associated with the specific disorder or pathologic condition; reduced morbidity and mortality, and improvement in quality-of-life issues. The above parameters for assessing successful treatment and improvement in the disorder are readily measurable by routine procedures familiar to a physician.

“U6 promoter” refers to the nucleic acids sequence that enable the control of small nuclear U6 RNA sequence transcription. An example of a nucleic acid sequence of U6 promotor is SEQ ID NO: 28.

“Virulence factor” refers to a micro-organism-derived molecule that contributes to its infectious power.

### DETAILED DESCRIPTION

The present invention relates to a polypeptide with cell penetrating capacity (i.e. a cell-penetrating polypeptide (CPP)) that is derived from a virulence factor. In some embodiments, the CPP comprises or consists of an amino acid sequence that is derived from a virulence factor or consists of a portion of a virulence factor. In some embodiments, the CPP is associated, or suitable to be associated, to a heterologous cargo

In some embodiments, the virulence factor is a Rho GTPase activator.

In some embodiments, the Cytotoxic Necrotizing Factor (CNF) family member is chosen among cytotoxic necrotizing factor 1 (CNF1), cytotoxic necrotizing factor 2 (CNF2), cytotoxic necrotizing factor 3 (CNF3), and cytotoxic necrotizing factor γ (CNFγ).

Cytotoxic necrotizing factor 1 (CNF1) is the most commonly described virulence factor from Escherichia coli. CNF1 is composed of a LRP 37 kDa receptor binding domain in N-terminal, a translocation domain, a Lu/BCAM receptor binding domain and a catalytic domain in C-terminal. The catalytic domain of CNF1 is released in the cytoplasm after acidification of the endosomal compartment through conformational changes of the translocation domain. CNF1 catalytic domain enables the deamidation of a glutamine residue that leads to activation of regulatory Rho, Rac and Cdc42 GTPases in eukaryotic cells controlling many processes such as organization and dynamics of actin cytoskeleton or DNA transcription. CNF1 is a 1014 amino acid protein (UniProt accession No Q47106—SEQ ID NO: 2). An example of a cDNA sequence encoding for CNF1 protein is SEQ ID NO: 1.

Cytotoxic necrotizing factor 2 (CNF2) is another Rho, Rac and Cdc42 GTPase activator virulence factor from Escherichia coli. CNF2 is a 1014 amino acid protein (UniProt accession No C5ZZQ2—SEQ ID NO: 4), put in evidence in cow fecal swabs. An example of cDNA sequence encoding for CNF2 protein is SEQ ID NO: 3.

Cytotoxic necrotizing factor 3 (CNF3) is another Rho, Rac and Cdc42 GTPase activator virulence factor from Escherichia coli. CNF3 is a 1013 amino acid protein (UniProt accession No Q0E668—SEQ ID NO: 6), put in evidence in sheep and goat. An example of cDNA sequence encoding for CNF3 protein is SEQ ID NO: 5.

Cytotoxic necrotizing factor γ (CNFγ) is a virulence factor from Yersinia pseudotuberculosis. CNFγ is a 1014 amino acid protein (UniProt accession No Q9EYH7-SEQ ID NO: 8), that has Rho GTPase activator activity as other CNFs from Escherichia coli but a higher substrate specificity for RhoA. An example of cDNA sequence encoding for CNFγ protein is SEQ ID NO: 7.

Preferably, the amino acid sequence that is derived from a portion of a virulence factor is at least 75%, 80%, 85%, 90%, 95%, 96%, 97%, 98%, 99% identical to said portion of the virulence factor.

In some embodiments, the cell penetrating polypeptide (CPP) comprises at least two different domains, at least three different domains, or at least four different domains. Preferably, the cell penetrating polypeptide (CPP) comprises three different domains.

In some embodiments, the CPP comprises or consists of, from N-terminus to C-terminus:
a first receptor binding domain of a virulence factor, and
one translocation domain of a virulence factor.

In some embodiments, the first receptor binding domain and the translocation domain are derived from different virulence factors. In other embodiments, the first receptor binding domain and the translocation domain are derived from a same virulence factor.

In some embodiments, the first receptor binding domain of a virulence factor is a Laminin Receptor binding domain, preferably the receptor binding domain of LRP 37 kDa. LRP 37 kDa is a cell surface laminin (basal lamina protein) receptor anchored in the plasma membrane. This receptor is known to be implicated in numerous physiological processes such as e. g. translation, maintenance of cytoskeletal structure, or cell differentiation, cell proliferation and cell migration. In some embodiments, the Laminin Receptor binding domain comprises or consists of the Laminin Receptor binding domain of CNF1, CNF2, CNF3 or CNFγ.

In some embodiments, the first receptor binding domain of a virulence factor comprises or consists of a sequence at least 60%, 65%, 70%, 75%, 80%, 85%, 90%, 95%, 96%, 97%, 98%, 99% identical, more preferably at least 85% identical, to SEQ ID NO: 9, SEQ ID NO: 12, SEQ ID NO: 15, or SEQ ID NO: 18, or a functional fragment thereof. Preferably, the first receptor binding domain comprised in the CPP exhibits a same function, or activity, as the domain from which it is derived, or from which it is a portion or fragment. For instance, the sequence at least 60%, 65%, 70%, 75%, 80%, 85%, 90%, 95%, 96%, 97%, 98%, 99% identical, more preferably at least 85% identical, to SEQ ID NO: 9, SEQ ID NO: 12, SEQ ID NO: 15, or SEQ ID NO: 18, or the functional fragment thereof, exhibits a same function, or activity, as SEQ ID NO: 9, SEQ ID NO: 12, SEQ ID NO: 15, or SEQ ID NO: 18, e.g. it allows specific cell targeting.

In some embodiments, the first receptor binding domain comprises or consists of an amino acid sequence as set forth in any one of SEQ ID NO: 9, SEQ ID NO: 12, SEQ ID NO: 15, or SEQ ID NO: 18, or a functional fragment thereof.

The amino acid sequences as set forth in any one of SEQ ID NO: 12, SEQ ID NO: 15, or SEQ ID NO: 18 have at least 60% identity with the amino acid sequence as set forth in SEQ ID NO: 9.

In some embodiments, the translocation domain is a PH sensitive domain that goes through conformational changes upon endosome acidification allowing translocation to the cytosol of the catalytic domain. Thus, one part of the domain becomes cytoplasmic whereas staying in the endosomal compartment after endocytosis. In some embodiments, the translocation domain comprises or consists of the translocation domain of CNF1, CNF2, CNF3, or CNFγ.

In other embodiments, the translocation domain is an acidic endosome-to-cytosol translocation domain sensitive to acidic pH that leads to a conformational change.

In some embodiment, the translocation domain comprises or consists of an amino acid sequence at least 60%, 65%, 70%, 75%, 80%, 85%, 90%, 95%, 96%, 97%, 98%, 99% identical, more preferably at least 85% identical, to SEQ ID NO: 10, SEQ ID NO: 13, SEQ ID NO: 16, or SEQ ID NO: 19, or a functional fragment thereof. Preferably, the translocation domain comprised in the CPP exhibits a same function, or activity, as the domain from which it is derived, or from which it is a portion or fragment. For instance, the sequence at least 60%, 65%, 70%, 75%, 80%, 85%, 90%, 95%, 96%, 97%, 98%, 99% identical, more preferably at least 85% identical, to SEQ ID NO: 10, SEQ ID NO: 13, SEQ ID NO: 16, or SEQ ID NO: 19, or the functional fragment thereof, exhibits a same function, or activity, as SEQ ID NO: 10, SEQ ID NO: 13, SEQ ID NO: 16, or SEQ ID NO: 19, e.g. it allows intracellular delivery.

In some embodiment, the translocation domain comprises or consists of an amino acid sequence as set forth in any one of SEQ ID NO: 10, SEQ ID NO: 13, SEQ ID NO: 16, or SEQ ID NO: 19, or a functional fragment thereof.

The amino acid sequences as set forth in any one of SEQ ID NO: 13, SEQ ID NO: 16, or SEQ ID NO: 19 have at least 60% identity with the amino acid sequence as set forth in SEQ ID NO: 10.

In some embodiments, the CPP does not comprise the catalytic domain of the virulence factor from which the CPP is derived. In some embodiments, the CPP does not comprise the catalytic domain of any virulence factor.

In some embodiments, the cell penetrating polypeptide (CPP) further comprises, from N-terminus to C-terminus, a second receptor binding domain.

In some embodiment, said second receptor binding domain is different from the first receptor binding domain.

In some embodiment, said second receptor binding domain is identical to the first receptor binding domain.

In some embodiment, the second receptor binding domain is the receptor binding domain to Lu/BCAM protein. Lutheran adhesion glycoprotein/basal cell adhesion molecules (Lu/BCAM) are transmembrane adhesion molecule members of the immunoglobulin family that act as laminin receptors and have been described as essential for CNF1 uptake by host cells. They differ by a length variation of their intracellular domains and are gathered under the abbreviation Lu/BCAM.

In some embodiments, the second receptor binding domain of a virulence factor comprises or consists of a sequence at least 60%, 65%, 70%, 75%, 80%, 85%, 90%, 95%, 96%, 97%, 98%, 99% identical, more preferably at least 85% identical, to SEQ ID NO: 11, SEQ ID NO: 14, or SEQ ID NO: 17, or a functional fragment thereof. Preferably, the second receptor binding domain comprised in the CPP exhibits a same function, or activity, as the domain from which it is derived, or from which it is a portion or fragment. For instance, the sequence at least 60%, 65%, 70%, 75%, 80%, 85%, 90%, 95%, 96%, 97%, 98%, 99% identical, more preferably at least 85% identical, to SEQ ID NO: 11, SEQ ID NO: 14, or SEQ ID NO: 17, or the functional fragment thereof, exhibits a same function, or activity, as SEQ ID NO: 11, SEQ ID NO: 14, or SEQ ID NO: 17, e.g. it allows specific cell targeting.

In some embodiment, the second receptor binding domain comprises or consists of an amino acid sequence as set forth in any one of SEQ ID NO: 11, SEQ ID NO: 14, or SEQ ID NO: 17, or a functional fragment thereof.

The amino acid sequences as set forth in any one of SEQ ID NO: 14 or SEQ ID NO: 17 have at least 60% identity with the amino acid sequence as set forth in SEQ ID NO: 11.

In some embodiment, the CPP of the invention comprises or consists of, from N-terminus to C-terminus:
a first receptor binding domain comprising or consisting of an amino acid sequence as set forth in any one of SEQ ID NO: 9, SEQ ID NO: 12, SEQ ID NO: 15, or SEQ ID NO: 18, or a functional fragment thereof as described hereabove; or an amino acid sequence at least 60%, 65%, 70%, 75%, 80%, 85%, 90%, 95%, 96%, 97%, 98%, 99% identical, more preferably at least 85% identical, to SEQ ID NO: 9, SEQ ID NO: 12, SEQ ID NO: 15, or SEQ ID NO: 18, or a functional fragment thereof; and
a translocation domain comprising or consisting of an amino acid sequence as set forth in any one of SEQ ID NO: 10, SEQ ID NO: 13, SEQ ID NO: 16, or SEQ ID NO: 19, or a functional fragment thereof, as described hereabove; or an amino acid sequence at least 60%, 65%, 70%, 75%, 80%, 85%, 90%, 95%, 96%, 97%, 98%, 99% identical, more preferably at least 85% identical, to SEQ ID NO: 10, SEQ ID NO: 13, SEQ ID NO: 16, or SEQ ID NO: 19, or a functional fragment thereof; and
optionally, a second receptor binding domain comprising or consisting of an amino acid sequence as set forth in any one of SEQ ID NO: 11, SEQ ID NO: 14, or SEQ ID NO: 17, or a functional fragment thereof, as described hereabove; or an amino acid sequence at least 60%, 65%, 70%, 75%, 80%, 85%, 90%, 95%, 96%, 97%, 98%, 99% identical, more preferably at least 85% identical, to SEQ ID NO: 11, SEQ ID NO: 14, or SEQ ID NO: 17, or a functional fragment thereof.

In some embodiment, the CPP of the invention comprises or consists of, from N-terminus to C-terminus:
a first receptor binding domain comprising or consisting of an amino acid sequence as set forth in any one of SEQ ID NO: 9, SEQ ID NO: 12, SEQ ID NO: 15, or SEQ ID NO: 18, or a functional fragment thereof; and
a translocation domain comprising or consisting of an amino acid sequence as set forth in any one SEQ ID NO: 10, SEQ ID NO: 13, SEQ ID NO: 16, or SEQ ID NO: 19, or a functional fragment thereof; and
optionally, a second receptor binding domain comprising or consisting of an amino acid sequence as set forth in any one of SEQ ID NO: 11, SEQ ID NO: 14, or SEQ ID NO: 17, or a functional fragment.

In another possible embodiment, the CPP has a sequence that is at least 55, 60, 65, 70, 75, 80, 85, 90, 95, preferably 99% identical to a CNF amino acid sequence exempt of its catalytic C-terminus domain.

In some embodiment, the CPP of the invention comprises or consists of, from N-terminus to C-terminus, any of the combinations of first receptor binding domain and translocation domain, or any of the combinations of first receptor binding domain, translocation domain and second receptor binding domain, as disclosed in Table 1.

TABLE 1

| Combination | First receptor binding | | Second receptor binding |
|---|---|---|---|
| no | domain | Translocation domain | domain |
| 1 | CNF-1 (SEQ ID NO: 9) | CNF-1 (SEQ ID NO: 10) | — |
| 2 | CNF-1 (SEQ ID NO: 9) | CNF-2 (SEQ ID NO: 13) | — |
| 3 | CNF-1 (SEQ ID NO: 9) | CNF-3 (SEQ ID NO: 16) | — |
| 4 | CNF-1 (SEQ ID NO: 9) | CNFγ (SEQ ID NO: 19) | — |
| 5 | CNF-2 (SEQ ID NO: 12) | CNF-1 (SEQ ID NO: 10) | — |
| 6 | CNF-2 (SEQ ID NO: 12) | CNF-2 (SEQ ID NO: 13) | — |
| 7 | CNF-2 (SEQ ID NO: 12) | CNF-3 (SEQ ID NO: 16) | — |
| 8 | CNF-2 (SEQ ID NO: 12) | CNFγ (SEQ ID NO: 19) | — |
| 9 | CNF-3 (SEQ ID NO: 15) | CNF-1 (SEQ ID NO: 10) | — |
| 10 | CNF-3 (SEQ ID NO: 15) | CNF-2 (SEQ ID NO: 13) | — |
| 11 | CNF-3 (SEQ ID NO: 15) | CNF-3 (SEQ ID NO: 16) | — |
| 12 | CNF-3 (SEQ ID NO: 15) | CNFγ (SEQ ID NO: 19) | — |
| 13 | CNFγ (SEQ ID NO: 18) | CNF-1 (SEQ ID NO: 10) | — |
| 14 | CNFγ (SEQ ID NO: 18) | CNF-2 (SEQ ID NO: 13) | — |
| 15 | CNFγ (SEQ ID NO: 18) | CNF-3 (SEQ ID NO: 16) | — |
| 16 | CNFγ (SEQ ID NO: 18) | CNFγ (SEQ ID NO: 19) | — |
| 17 | CNF-1 (SEQ ID NO: 9) | CNF-1 (SEQ ID NO: 10) | CNF-1 (SEQ ID NO: 11) |
| 18 | CNF-1 (SEQ ID NO: 9) | CNF-1 (SEQ ID NO: 10) | CNF-2 (SEQ ID NO: 14) |
| 19 | CNF-1 (SEQ ID NO: 9) | CNF-1 (SEQ ID NO: 10) | CNF-3 (SEQ ID NO: 17) |
| 20 | CNF-1 (SEQ ID NO: 9) | CNF-2 (SEQ ID NO: 13) | CNF-1 (SEQ ID NO: 11) |
| 21 | CNF-1 (SEQ ID NO: 9) | CNF-2 (SEQ ID NO: 13) | CNF-2 (SEQ ID NO: 14) |
| 22 | CNF-1 (SEQ ID NO: 9) | CNF-2 (SEQ ID NO: 13) | CNF-3 (SEQ ID NO: 17) |
| 23 | CNF-1 (SEQ ID NO: 9) | CNF-3 (SEQ ID NO: 16) | CNF-1 (SEQ ID NO: 11) |
| 24 | CNF-1 (SEQ ID NO: 9) | CNF-3 (SEQ ID NO: 16) | CNF-2 (SEQ ID NO: 14) |
| 25 | CNF-1 (SEQ ID NO: 9) | CNF-3 (SEQ ID NO: 16) | CNF-3 (SEQ ID NO: 17) |
| 26 | CNF-1 (SEQ ID NO: 9) | CNFγ (SEQ ID NO: 19) | CNF-1 (SEQ ID NO: 11) |
| 27 | CNF-1 (SEQ ID NO: 9) | CNFγ (SEQ ID NO: 19) | CNF-2 (SEQ ID NO: 14) |
| 28 | CNF-1 (SEQ ID NO: 9) | CNFγ (SEQ ID NO: 19) | CNF-3 (SEQ ID NO: 17) |
| 29 | CNF-2 (SEQ ID NO: 12) | CNF-1 (SEQ ID NO: 10) | CNF-1 (SEQ ID NO: 11) |
| 30 | CNF-2 (SEQ ID NO: 12) | CNF-1 (SEQ ID NO: 10) | CNF-2 (SEQ ID NO: 14) |
| 31 | CNF-2 (SEQ ID NO: 12) | CNF-1 (SEQ ID NO: 10) | CNF-3 (SEQ ID NO: 17) |
| 32 | CNF-2 (SEQ ID NO: 12) | CNF-2 (SEQ ID NO: 13) | CNF-1 (SEQ ID NO: 11) |
| 33 | CNF-2 (SEQ ID NO: 12) | CNF-2 (SEQ ID NO: 13) | CNF-2 (SEQ ID NO: 14) |
| 34 | CNF-2 (SEQ ID NO: 12) | CNF-2 (SEQ ID NO: 13) | CNF-3 (SEQ ID NO: 17) |
| 35 | CNF-2 (SEQ ID NO: 12) | CNF-3 (SEQ ID NO: 16) | CNF-1 (SEQ ID NO: 11) |
| 36 | CNF-2 (SEQ ID NO: 12) | CNF-3 (SEQ ID NO: 16) | CNF-2 (SEQ ID NO: 14) |
| 37 | CNF-2 (SEQ ID NO: 12) | CNF-3 (SEQ ID NO: 16) | CNF-3 (SEQ ID NO: 17) |
| 38 | CNF-2 (SEQ ID NO: 12) | CNFγ (SEQ ID NO: 19) | CNF-1 (SEQ ID NO: 11) |
| 39 | CNF-2 (SEQ ID NO: 12) | CNFγ (SEQ ID NO: 19) | CNF-2 (SEQ ID NO: 14) |
| 40 | CNF-2 (SEQ ID NO: 12) | CNFγ (SEQ ID NO: 19) | CNF-3 (SEQ ID NO: 17) |
| 41 | CNF-3 (SEQ ID NO: 15) | CNF-1 (SEQ ID NO: 10) | CNF-1 (SEQ ID NO: 11) |
| 42 | CNF-3 (SEQ ID NO: 15) | CNF-1 (SEQ ID NO: 10) | CNF-2 (SEQ ID NO: 14) |
| 43 | CNF-3 (SEQ ID NO: 15) | CNF-1 (SEQ ID NO: 10) | CNF-3 (SEQ ID NO: 17) |
| 44 | CNF-3 (SEQ ID NO: 15) | CNF-2 (SEQ ID NO: 13) | CNF-1 (SEQ ID NO: 11) |
| 45 | CNF-3 (SEQ ID NO: 15) | CNF-2 (SEQ ID NO: 13) | CNF-2 (SEQ ID NO: 14) |
| 46 | CNF-3 (SEQ ID NO: 15) | CNF-2 (SEQ ID NO: 13) | CNF-3 (SEQ ID NO: 17) |
| 47 | CNF-3 (SEQ ID NO: 15) | CNF-3 (SEQ ID NO: 16) | CNF-1 (SEQ ID NO: 11) |
| 48 | CNF-3 (SEQ ID NO: 15) | CNF-3 (SEQ ID NO: 16) | CNF-2 (SEQ ID NO: 14) |
| 49 | CNF-3 (SEQ ID NO: 15) | CNF-3 (SEQ ID NO: 16) | CNF-3 (SEQ ID NO: 17) |
| 50 | CNF-3 (SEQ ID NO: 15) | CNFγ (SEQ ID NO: 19) | CNF-1 (SEQ ID NO: 11) |
| 51 | CNF-3 (SEQ ID NO: 15) | CNFγ (SEQ ID NO: 19) | CNF-2 (SEQ ID NO: 14) |
| 52 | CNF-3 (SEQ ID NO: 15) | CNFγ (SEQ ID NO: 19) | CNF-3 (SEQ ID NO: 17) |
| 53 | CNFγ (SEQ ID NO: 18) | CNF-1 (SEQ ID NO: 10) | CNF-1 (SEQ ID NO: 11) |
| 54 | CNFγ (SEQ ID NO: 18) | CNF-1 (SEQ ID NO: 10) | CNF-2 (SEQ ID NO: 14) |
| 55 | CNFγ (SEQ ID NO: 18) | CNF-1 (SEQ ID NO: 10) | CNF-3 (SEQ ID NO: 17) |
| 56 | CNFγ (SEQ ID NO: 18) | CNF-2 (SEQ ID NO: 13) | CNF-1 (SEQ ID NO: 11) |
| 57 | CNFγ (SEQ ID NO: 18) | CNF-2 (SEQ ID NO: 13) | CNF-2 (SEQ ID NO: 14) |
| 58 | CNFγ (SEQ ID NO: 18) | CNF-2 (SEQ ID NO: 13) | CNF-3 (SEQ ID NO: 17) |
| 59 | CNFγ (SEQ ID NO: 18) | CNF-3 (SEQ ID NO: 16) | CNF-1 (SEQ ID NO: 11) |
| 60 | CNFγ (SEQ ID NO: 18) | CNF-3 (SEQ ID NO: 16) | CNF-2 (SEQ ID NO: 14) |
| 61 | CNFγ (SEQ ID NO: 18) | CNF-3 (SEQ ID NO: 16) | CNF-3 (SEQ ID NO: 17) |
| 62 | CNFγ (SEQ ID NO: 18) | CNFγ (SEQ ID NO: 19) | CNF-1 (SEQ ID NO: 11) |
| 63 | CNFγ (SEQ ID NO: 18) | CNFγ (SEQ ID NO: 19) | CNF-2 (SEQ ID NO: 14) |
| 64 | CNFγ (SEQ ID NO: 18) | CNFγ (SEQ ID NO: 19) | CNF-3 (SEQ ID NO: 17) |

In some embodiments, the CPP of the invention comprises or consists of an amino acid sequence as set forth in SEQ ID NO: 20, SEQ ID NO: 22, SEQ ID NO: 24, or SEQ ID NO: 26, or a fragment thereof; or an amino acid sequence having at least 60%, 65% 70%, 75%, 80%, 85%, 90%, 91%, 92%, 93%, 94%, 95%, 96%, 97%, 98% or 99% or more of identity with the amino acid sequence as set forth in SEQ ID NO: 20, SEQ ID NO: 22, SEQ ID NO: 24, or SEQ ID NO: 26, or a fragment thereof.

Preferably, the sequence at least 60%, 65%, 70%, 75%, 80%, 85%, 90%, 95%, 96%, 97%, 98%, 99% identical, more preferably at least 85% identical, to SEQ ID NO: 20, SEQ ID NO: 22, SEQ ID NO: 24, or SEQ ID NO: 26, or the functional fragment thereof, exhibits a same function, or activity, as SEQ ID NO: 20, SEQ ID NO: 22, SEQ ID NO: 24, or SEQ ID NO: 26, e.g. it allows specific cell targeting and intracellular delivery.

The present invention also relates to a complex comprising the cell penetrating polypeptide (CPP) according to the invention associated, e.g. conjugated, to at least one cargo.

The CPP may be associated to the cargo via a linker or not. In some embodiments, the complex comprising the cell penetrating polypeptide (CPP) according to the invention is conjugated to at least one cargo through a linker.

The CPP may be associated to the cargo by complementary base matching, covalent bond, ionic bond, hydrogen bond, polar bond, hydrophobic interaction or effect (van der Waals force), electrostatic interaction or forces (e.g. between a RNA or DNA and a protein), biotin/streptavidin interaction, and the like. In some embodiments, the CPP is associated to the cargo by a covalent bond.

For instance, the cargo may be a single domain antibody associated to the CPP by a covalent bond.

In another example, the cargo may be a CRISPR associated protein (e.g. Cas9) associated to the CPP by a covalent bond. In some embodiments, the CRISPR associated protein is further associated to a sgRNA (guide RNA) by electrostatic interactions.

In another example, the cargo may be a sgRNA (guide RNA) associated to the CPP by electrostatic interactions.

In another example, the cargo may be a biotinylated protein, polynucleotide or ribonucleotide associated to the CPP by a covalent bond. In some embodiments, the biotinylated protein further interacts with a streptavidin fusion protein.

All the embodiments detailed in the preceding section in connection with the first aspect of the invention are also preferred embodiments according to this aspect of the invention.

Methods for non-covalently conjugating a peptide or polypeptide to a molecule of interest are well-known. Methods for covalently conjugating a peptide or polypeptide to a molecule of interest are well-known.

In some embodiments, the cargo comprises or consists of at least one molecule of interest, which is associated, e.g. conjugated, to the CPP. Preferably, the at least one molecule of interest is associated, e.g. conjugated, to the C-terminus of the CPP.

In some embodiments, the cargo is not derived from a virulence factor, in particular from a CNF virulence factor.

In some embodiments, the cargo comprises at least one molecule of interest or a functionally and/or structurally active fragment thereof. In particular, the molecule of interest may be a nucleic acid molecule, an amino acid molecule, a therapeutically active peptide or protein, a protein, a fluorescent protein, an antibody, a ribonucleoprotein, an enzyme, a transcription factor, a carbohydrate, a lipid, a glycan, a contrast or imaging agent, a quantum dot, a diagnostic agent, a therapeutic agent, and any combination thereof.

In some embodiment, the cargo comprises or consists of at least 1, 2, 3, 4, 5, 6, or more molecules of interest chosen in the group consisting of: a nucleic acid molecule, an amino acid molecule, a therapeutically active peptide or protein, a protein, a fluorescent protein, an antibody, a ribonucleoprotein, an enzyme, a transcription factor, a carbohydrate, a lipid, a glycan, a contrast or imaging agent, a quantum dot, a diagnostic agent, a therapeutic agent, and any combination thereof.

For instance, the cargo may be:
a molecule used in the CRISPR/Cas system such as e.g. a CRISPR associated protein and/or a sgRNA (guide RNA);
a ribonucleoprotein (allowing RNA or DNA delivery);
a single chain antibody (scFv) or a single domain antibody (such as a nanobody),
a lysosomal enzyme or polypeptide;
a ubiquitin ligase; and/or
a transcription factor.

In some embodiment, the molecule of interest comprises or consists of an amino acid sequence, for example a peptide, a polypeptide, a protein, a fluorescent protein, an enzyme, a linker, an addressing cellular localization sequence (e. g. MTS, NLS . . . ), etc. The specific combination of CPP and an amino acid sequence is called a fusion protein or a fusion polypeptide.

In other embodiments, the molecule of interest comprises or consists of a deoxyribonucleic acid (DNA) or a complementary DNA (cDNA).

In other embodiments, the molecule of interest comprises or consists of a ribonucleic acid (RNA). The RNA could be of any known types, e. g. small interfering RNA (siRNA), short hairpin RNA (shRNA), single guide RNA (sgRNA), transfer RNA (tRNA), etc.

In other embodiments, the molecule of interest comprises or consists of an antibody. This antibody may be a combination of two heavy chains and two light chains or a fragment thereof. In some embodiment, the antibody is a scFv or a nanobody.

In other embodiments, the molecule of interest comprises or consists of a therapeutic molecule, preferably an active therapeutic molecule or an antibody, a ribonucleoprotein, an enzyme, a transcription factor, a carbohydrate, a lipid, a glycan, a contrast or imaging agent, a quantum dot, a diagnostic agent, a therapeutic agent, or any combination of thereof.

In some embodiments, the cargo comprises or consists of at least 2 molecules of interest, such as, for example, one amino acid sequence and one nucleic acid sequence, including both DNA and/or RNA type. In some embodiment, the DNA is a cDNA. In some embodiment, the RNA is any kind of RNA sequence, for example sgRNA (single guide), siRNA (small interfering), shRNA (short hairpin), mRNA (messenger), tRNA (transfer), etc.

In a preferred embodiment, the cargo comprises or consists of a combination of molecules of interest comprising or consisting of a CRISPR associated protein amino acid sequence and a sgRNA. Preferably, said CRISPR associated protein amino acid sequence is a Cas9 or Cas12 sequence. Examples of Cas9 and Cas12 sequences are those disclosed in Shmakov et al. 2017 or those defined by amino acid sequences SEQ ID NO: 44 or SEQ ID NO: 46 or by nucleic acid sequences SEQ ID NO: 45 or SEQ ID NO: 47.

The present invention relates to a polynucleotide or nucleic acid, or a set of polynucleotides or nucleic acids, encoding the CPP according to the invention or encoding the fusion polypeptide or encoding and/or comprising the complex or a portion of the complex (comprising the CPP and a portion of the heterologous cargo) according to the invention.

All the embodiments detailed in the preceding section in connection with the first aspect of the invention are also preferred embodiments according to this aspect of the invention.

In some embodiments, the polynucleotide of the invention encodes a CPP comprising or consisting of, from N-terminus to C-terminus, any of the combinations of first receptor binding domain and translocation domain, or any of the combinations of first receptor binding domain, translocation domain and second receptor binding domain, as disclosed in Table 1.

In some embodiments, the polynucleotide of the invention comprises or consists of nucleic acid sequence at least 60%, 65%, 70%, 75%, 80%, 85%, 90%, 95%, 96%, 97%, 98%, 99% identical, more preferably at least 85% identical, to SEQ ID NO: 21, SEQ ID NO: 23, SEQ ID NO: 25, or SEQ ID NO: 27, or a fragment thereof.

In some embodiments, the polynucleotide of the invention comprises or consists of nucleic acid sequence as set forth in any one of SEQ ID NO: 21, SEQ ID NO: 23, SEQ ID NO: 25, or SEQ ID NO: 27, or a fragment thereof.

In a preferred embodiment, the polynucleotide of the invention comprises or consists of nucleic acid sequence as set forth in sequence SEQ ID NO: 21, or a fragment thereof.

In some embodiments, the polynucleotide or set of polynucleotides of the invention encodes a fusion polypeptide comprising or consisting of:
a CPP amino acid sequence comprising or consisting of, from N-terminus to C-terminus, any of the combinations of first receptor binding domain and translocation domain, or any of the combinations of first receptor binding domain, translocation domain and second receptor binding domain, as disclosed in Table 1, or a fragment thereof, and
at least 1, 2, 3, 4, 5, or 6 molecules of interest chosen in the group consisting of: a nucleic acid molecule, an amino acid molecule, a therapeutically active peptide or protein, a protein, an antibody, a ribonucleoprotein, an enzyme, a transcription factor, a contrast or imaging agent, a diagnostic agent, a therapeutic agent, and any combination thereof.

In some embodiments, the polynucleotide or set of polynucleotides of the invention encodes a fusion polypeptide comprising or consisting of:
a CPP amino acid sequence as set forth in SEQ ID NO: 20, SEQ ID NO: 22, SEQ ID NO: 24, or SEQ ID NO: 26, or a fragment thereof; or encodes an amino acid sequence having at least 60%, 65% 70%, 75%, 80%, 85%, 90%, 91%, 92%, 93%, 94%, 95%, 96%, 97%, 98% or 99% or more identity with the amino acid sequence as set forth in SEQ ID NO: 20, SEQ ID NO: 22, SEQ ID NO: 24, or SEQ ID NO: 26, or a fragment thereof, and
at least 1, 2, 3, 4, 5, or 6 molecules of interest chosen in the group consisting of: a nucleic acid molecule, an amino acid molecule, a therapeutically active peptide or protein, a protein, an antibody, a ribonucleoprotein, an enzyme, a transcription factor, a contrast or imaging agent, a diagnostic agent, a therapeutic agent, and any combination thereof.

In some preferred embodiment, the polynucleotide or set of polynucleotides of the invention encodes a fusion polypeptide consisting of or comprising:
a CPP amino acid sequence as set forth in SEQ ID NO: 20, SEQ ID NO: 22, SEQ ID NO: 24, or SEQ ID NO: 26, or a fragment thereof; or encodes an amino acid sequence having at least 60%, 65% 70%, 75%, 80%, 85%, 90%, 91%, 92%, 93%, 94%, 95%, 96%, 97%, 98% or 99% or more identity with the amino acid sequence as set forth in SEQ ID NO: 20, SEQ ID NO: 22, SEQ ID NO: 24, or SEQ ID NO: 26, or a fragment thereof, and
a CRISPR associated protein, preferably Cas9 or Cas12.

In some embodiments, the polynucleotide or set of polynucleotides of the invention encodes and/or comprises a complex of the invention or a portion of a complex of the invention comprising or consisting of:
a CPP amino acid sequence comprising or consisting of, from N-terminus to C-terminus, any of the combinations of first receptor binding domain and translocation domain, or any of the combinations of first receptor binding domain, translocation domain and second receptor binding domain, as disclosed in Table 1, or a fragment thereof, and
at least 1, 2, 3, 4, 5, or 6 molecules of interest chosen in the group consisting of: a nucleic acid molecule, an amino acid molecule, a therapeutically active peptide or protein, a protein, an antibody, a contrast or imaging agent, a diagnostic agent, a therapeutic agent, and any combination thereof.

In some preferred embodiment, the polynucleotide or set of polynucleotides of the invention encodes and/or comprises a complex of the invention or a portion of a complex of the invention comprising or consisting of:
a CPP amino acid sequence as set forth in SEQ ID NO: 20, SEQ ID NO: 22, SEQ ID NO: 24, or SEQ ID NO: 26, or a fragment thereof; or encodes an amino acid sequence having at least 60%, 65% 70%, 75%, 80%, 85%, 90%, 91%, 92%, 93%, 94%, 95%, 96%, 97%, 98% or 99% or more identity with the amino acid sequence as set forth in SEQ ID NO: 20, SEQ ID NO: 22, SEQ ID NO: 24, or SEQ ID NO: 26, or a fragment thereof, and
at least 1, 2, 3, 4, 5, or 6 molecules of interest chosen in the group consisting of: a nucleic acid molecule, an amino acid molecule, a therapeutically active peptide or protein, a protein, an antibody, a contrast or imaging agent, a diagnostic agent, a therapeutic agent, and any combination thereof.

In some preferred embodiment, the polynucleotide or set of polynucleotides of the invention encodes and/or comprises a complex of the invention or a portion of a complex of the invention consisting of or comprising:
a CPP amino acid sequence as set forth in SEQ ID NO: 20, SEQ ID NO: 22, SEQ ID NO: 24, or SEQ ID NO: 26, or a fragment thereof; or encodes an amino acid sequence having at least 60%, 65% 70%, 75%, 80%, 85%, 90%, 91%, 92%, 93%, 94%, 95%, 96%, 97%, 98% or 99% or more identity with the amino acid sequence as set forth in SEQ ID NO: 20, SEQ ID NO: 22, SEQ ID NO: 24, or SEQ ID NO: 26, or a fragment thereof, and
a CRISPR associated protein, preferably Cas9 or Cas12, and/or a sgRNA.

The various parts of the complex, i.e. the CPP and the various parts of the cargo, may be encoded by, or may consist of, one or several polynucleotides.

For instance, the set of polynucleotides of the invention may comprise 1, 2, 3, 4, 5, 6 or more polynucleotides.

According to the present invention, the nucleic acid sequence of the invention encoding CPP as described hereinabove, may be any nucleic acid sequence that is a degenerate version of a nucleic acid sequence and that encodes the same CPP.

One skilled in the art is familiar with methods for adapting a coding sequence based on the genetic code, for instance and without limitation, methods making use of codon degeneracy to introduce silent mutations and methods taking into account codon usage bias and variation of the standard genetic code relevant to the host cell considered.

The present invention also relates to a vector, in particular a recombinant expression vector, comprising the polynucleotide of the invention as described hereinabove. Examples of vectors include, without being limited to, plasmids, viral vectors, artificial chromosomes, liposomes, and lipid nanoparticles.

In some embodiments, the vector is a nucleic acid molecule as described herein.

In some embodiments, the vector as described herein comprises a nucleotide sequence at least 60%, 65%, 70%, 75%, 80%, 85%, 90%, 95%, 96%, 97%, 98%, 99% or more, more preferably at least 85% identical to SEQ ID NO: 21, SEQ ID NO: 23, SEQ ID NO: 25, or SEQ ID NO: 27, or a fragment thereof.

In some embodiments, the vector as described herein comprises a nucleotide sequence as set forth in any one of sequences SEQ ID NO: 21, SEQ ID NO: 23, SEQ ID NO: 25, or SEQ ID NO: 27, or a fragment thereof.

In some embodiments, the vector as described herein comprises a nucleotide sequence encoding a CPP comprising or consisting of, from N-terminus to C-terminus, any of the combinations of first receptor binding domain and translocation domain, or any of the combinations of first receptor binding domain, translocation domain and second receptor binding domain, as disclosed in Table 1 above.

In some embodiments, the vector as described herein comprises a nucleotide sequence encoding a fusion polypeptide comprising or consisting of:
a CPP amino acid sequence comprising or consisting of, from N-terminus to C-terminus, any of the combinations of first receptor binding domain and translocation domain, or any of the combinations of first receptor binding domain, translocation domain and second receptor binding domain, as disclosed in Table 1, or a fragment thereof, and
at least 1, 2, 3, 4, 5, or 6 molecules of interest chosen in the group consisting of: a nucleic acid molecule, an amino acid molecule, a therapeutically active peptide or protein, a protein, an antibody, a ribonucleoprotein, an enzyme, a transcription factor, a contrast or imaging agent, a diagnostic agent, a therapeutic agent, and any combination thereof.

In some embodiments, the vector as described herein comprises a nucleotide sequence encoding a fusion polypeptide comprising or consisting of:
a CPP amino acid sequence as set forth in SEQ ID NO: 20, SEQ ID NO: 22, SEQ ID NO: 24, or SEQ ID NO: 26, or a fragment thereof; or encodes an amino acid sequence having at least 60%, 65% 70%, 75%, 80%, 85%, 90%, 91%, 92%, 93%, 94%, 95%, 96%, 97%, 98%, or 99% or more identity with the amino acid sequence as set forth in SEQ ID NO: 20, SEQ ID NO: 22, SEQ ID NO: 24, or SEQ ID NO: 26, or a fragment thereof, and
at least 1, 2, 3, 4, 5, or 6 molecules of interest chosen in the group consisting of: a nucleic acid molecule, an amino acid molecule, a therapeutically active peptide or protein, a protein, an antibody, a ribonucleoprotein, an enzyme, a transcription factor, a contrast or imaging agent, a diagnostic agent, a therapeutic agent, and any combination thereof.

In some embodiments, the vector as described herein comprises a nucleotide sequence encoding a fusion polypeptide comprising or consisting of:
a CPP amino acid sequence as set forth in SEQ ID NO: 20, SEQ ID NO: 22, SEQ ID NO: 24, or SEQ ID NO: 26, or a fragment thereof; or encodes an amino acid sequence having at least 60%, 65% 70%, 75%, 80%, 85%, 90%, 91%, 92%, 93%, 94%, 95%, 96%, 97%, 98%, or 99% or more identity with the amino acid sequence as set forth in SEQ ID NO: 20, SEQ ID NO: 22, SEQ ID NO: 24, or SEQ ID NO: 26, or a fragment thereof, and
a CRISPR associated protein, preferably Cas9 or Cas12.

In some embodiments, the vector as described herein comprises a nucleotide sequence encoding and/or comprising a complex of the invention or a portion of a complex of the invention comprising or consisting of:
a CPP amino acid sequence comprising or consisting of, from N-terminus to C-terminus, any of the combinations of first receptor binding domain and translocation domain, or any of the combinations of first receptor binding domain, translocation domain and second receptor binding domain, as disclosed in Table 1, or a fragment thereof, and
at least 1, 2, 3, 4, 5, or 6 molecules of interest chosen in the group consisting of: a nucleic acid molecule, an amino acid molecule, a therapeutically active peptide or protein, a protein, an antibody, a contrast or imaging agent, a diagnostic agent, a therapeutic agent, and any combination thereof.

In some preferred embodiment, the vector as described herein comprises a nucleotide sequence encoding and/or comprising a complex of the invention or a portion of a complex of the invention comprising or consisting of:
a CPP amino acid sequence as set forth in SEQ ID NO: 20, SEQ ID NO: 22, SEQ ID NO: 24, or SEQ ID NO: 26, or a fragment thereof; or encodes an amino acid sequence having at least 60%, 65% 70%, 75%, 80%, 85%, 90%, 91%, 92%, 93%, 94%, 95%, 96%, 97%, 98% or 99% or more identity with the amino acid sequence as set forth in SEQ ID NO: 20, SEQ ID NO: 22, SEQ ID NO: 24, or SEQ ID NO: 26, or a fragment thereof, and
at least 1, 2, 3, 4, 5, or 6 molecules of interest chosen in the group consisting of: a nucleic acid molecule, an amino acid molecule, a therapeutically active peptide or protein, a protein, an antibody, a contrast or imaging agent, a diagnostic agent, a therapeutic agent, and any combination thereof.

In some preferred embodiment, the vector as described herein comprises a nucleotide sequence encoding and/or comprising a complex of the invention or a portion of a complex of the invention consisting of or comprising:
a CPP amino acid sequence as set forth in SEQ ID NO: 20, SEQ ID NO: 22, SEQ ID NO: 24, or SEQ ID NO: 26, or a fragment thereof; or encodes an amino acid sequence having at least 60%, 65% 70%, 75%, 80%, 85%, 90%, 91%, 92%, 93%, 94%, 95%, 96%, 97%, 98% or 99% or more identity with the amino acid sequence as set forth in SEQ ID NO: 20, SEQ ID NO: 22, SEQ ID NO: 24, or SEQ ID NO: 26, or a fragment thereof, and
a CRISPR associated protein, preferably Cas9 or Cas12, and/or a sgRNA.

The various parts of the complex, i.e. the CPP and the various parts of the cargo, may be encoded by, or may consist of, one or several nucleotide sequence(s).

In some embodiment, the vector of the invention further comprises a nucleic acid sequence encoding an Optimized sequence of CNF1 Delivery Mechanism (OCDM) as set forth in SEQ ID NO: 21.

One skilled in the art is familiar with nucleic acid sequence that are necessary to build a vector. The expression vector may comprise a replication origin site and the sequence of an antibiotic resistance gene under appropriate promoter control to select the cells that correctly express the vector. Non limited examples of expression vectors are plasmids, cosmids, fosmids and the like.

In some embodiments, the OCDM sequence is controlled by a viral promotor, preferably a cytomegalovirus (CMV) promoter.

In some embodiments, the vector further comprises the coding sequence of a CRISPR associated protein followed by nuclear localization sequence (NLS) encoding sequence under the same promotor of OCDM to be part of the same transcript.

In some embodiments, the sgRNA encoding sequence is under the control of a U6 (gene) promotor.

In some embodiments, the vector comprises a Flag tag sequence inserted before and/or after an OCDM sequence and/or molecules of interest coding sequence.

In some embodiments, the expression vector comprises a nucleic acid sequence encoding an antibody. In some embodiments the nucleic acid sequence encoding antibody consists of a sequence encoding a single chain antibody, or a nanobody, under the same promotor of OCDM in order to be part of the same transcript.

The present invention also concerns a cell, cell population or cell line comprising or expressing the CPP according to the invention, a complex according to the invention, a fusion polypeptide according to the invention, a polynucleotide according to the invention, or a vector according to the invention.

All the embodiments detailed in the preceding section in connection with the first aspect of the invention are also preferred embodiments according to this aspect of the invention.

The cell or cell line as described herein may be a genetically modified cell, cell population or cell line, that is to say a cell or cell line genetically modified to express the CPP according to the invention, the fusion polypeptide according to the invention, the polynucleotide according to the invention, or the vector according to the invention.

The cell, cell population or cell line is preferably a eukaryotic cell or cell line. The cell, cell population or cell line may be an animal cell, cell population or cell line. The cell, cell population or cell line may be a mammal cell, cell population or cell line. The cell, cell population or cell line may be a human cell, cell population or cell line. The cell, cell population or cell line may be a primary cell, cell population or cell line, in particular a human primary cell, cell population or cell line. The cell, cell population or cell line may be an immortalized cell, cell population or cell line, in particular a human immortalized cell, cell population or cell line. The cell, cell population or cell line may be an immune cell, cell population or cell line, in particular a human immune cell, cell population or cell line.

In some embodiments, the cells are Human embryonic kidney 293, in particular 293 cells expressing simian virus (SV40) tsA1609 large T antigen allele (HEK293T), or Chinese Hamster Ovary (CHO) cells.

In some embodiments, the cell, cell population or cell line as described herein comprises or expresses a vector as described herein.

In some embodiments, the cell or cell line as described herein comprises or expresses at least one CPP according to the invention, fusion polypeptide or complex according to the invention.

In some embodiments, the cell or cell line as described herein comprises or expresses at least one fusion polypeptide comprising or consisting of:
a CPP amino acid sequence comprising or consisting of, from N-terminus to C-terminus, any of the combinations of first receptor binding domain and translocation domain, or any of the combinations of first receptor binding domain, translocation domain and second receptor binding domain, as disclosed in Table 1, or a fragment thereof, and
at least 1, 2, 3, 4, 5, or 6 molecules of interest chosen in the group consisting of: a nucleic acid molecule, an amino acid molecule, a therapeutically active peptide or protein, a protein, an antibody, a ribonucleoprotein, an enzyme, a transcription factor, a contrast or imaging agent, a diagnostic agent, a therapeutic agent, and any combination thereof.

In some embodiments, the cell or cell line as described herein comprises or expresses at least one fusion polypeptide comprising or consisting of:
a CPP amino acid sequence as set forth in SEQ ID NO: 20, SEQ ID NO: 22, SEQ ID NO: 24, or SEQ ID NO: 26, or a fragment thereof; or encodes an amino acid sequence having at least 60%, 65% 70%, 75%, 80%, 85%, 90%, 91%, 92%, 93%, 94%, 95%, 96%, 97%, 98%, or 99% or more identity with the amino acid sequence as set forth in SEQ ID NO: 20, SEQ ID NO: 22, SEQ ID NO: 24, SEQ ID NO: 26, or a fragment thereof, and
at least 1, 2, 3, 4, 5, or 6 molecules of interest chosen in the group consisting of: a nucleic acid molecule, an amino acid molecule, a therapeutically active peptide or protein, a protein, an antibody, a ribonucleoprotein, an enzyme, a transcription factor, a contrast or imaging agent, a diagnostic agent, a therapeutic agent, and any combination thereof.

In some embodiments, the cell or cell line as described herein comprises or expresses at least one fusion polypeptide comprising or consisting of:
a CPP amino acid sequence as set forth in SEQ ID NO: 20, SEQ ID NO: 22, SEQ ID NO: 24, SEQ ID NO: 26, or a fragment thereof; or encodes an amino acid sequence having at least 60%, 65% 70%, 75%, 80%, 85%, 90%, 91%, 92%, 93%, 94%, 95%, 96%, 97%, 98%, or 99% or more identity with the amino acid sequence as set forth in SEQ ID NO: 20, SEQ ID NO: 22, SEQ ID NO: 24, SEQ ID NO: 26, or a fragment thereof, and
a CRISPR associated protein, preferably Cas9 or Cas12.

In some embodiments, the cell or cell line as described herein comprises or expresses at least one complex of the invention or portion of a complex of the invention comprising or consisting of:
a CPP amino acid sequence comprising or consisting of, from N-terminus to C-terminus, any of the combinations of first receptor binding domain and translocation domain, or any of the combinations of first receptor binding domain, translocation domain and second receptor binding domain, as disclosed in Table 1, or a fragment thereof, and
at least 1, 2, 3, 4, 5, or 6 molecules of interest chosen in the group consisting of: a nucleic acid molecule, an amino acid molecule, a therapeutically active peptide or protein, a protein, an antibody, a contrast or imaging agent, a diagnostic agent, a therapeutic agent, and any combination thereof.

In some preferred embodiment, the cell or cell line as described herein comprises or expresses at least one complex of the invention or portion of a complex of the invention comprising or consisting of:
a CPP amino acid sequence as set forth in SEQ ID NO: 20, SEQ ID NO: 22, SEQ ID NO: 24, or SEQ ID NO: 26, or a fragment thereof; or encodes an amino acid sequence having at least 60%, 65% 70%, 75%, 80%, 85%, 90%, 91%, 92%, 93%, 94%, 95%, 96%, 97%, 98% or 99% or more identity with the amino acid sequence as set forth in SEQ ID NO: 20, SEQ ID NO: 22, SEQ ID NO: 24, or SEQ ID NO: 26, or a fragment thereof, and
at least 1, 2, 3, 4, 5, or 6 molecules of interest chosen in the group consisting of: a nucleic acid molecule, an amino acid molecule, a therapeutically active peptide or protein, a protein, an antibody, a contrast or imaging agent, a diagnostic agent, a therapeutic agent, and any combination thereof.

In some preferred embodiment, the cell or cell line as described herein comprises or expresses at least one complex of the invention or portion of a complex of the invention comprising or consisting of:
a CPP amino acid sequence as set forth in SEQ ID NO: 20, SEQ ID NO: 22, SEQ ID NO: 24, or SEQ ID NO: 26, or a fragment thereof; or encodes an amino acid sequence having at least 60%, 65% 70%, 75%, 80%, 85%, 90%, 91%, 92%, 93%, 94%, 95%, 96%, 97%, 98% or 99% or more identity with the amino acid sequence as set forth in SEQ ID NO: 20, SEQ ID NO: 22, SEQ ID NO: 24, or SEQ ID NO: 26, or a fragment thereof, and
a CRISPR associated protein, preferably Cas9 or Cas12, and/or a sgRNA.

In some embodiments, the CPP according to the invention or the fusion polypeptide according to the invention are produced in mammalian cells and purified through known method from the person of the skill, for instance by means of beads able to catch specific peptide of the complex. Beads are classically agarose beads coated with antibody that can target a specific antigen. Those beads enable immunoprecipitation of a complex containing the antigen and can be isolated for example through magnetism or centrifugation.

In some embodiments, the specific peptide targeted by the beads is a flag tag.

The invention is further directed to an in vitro method for directing or delivering a molecule of interest into cells, cell populations or cell lines, or a method for delivering at least one molecule of interest into cells, cell population or cell lines, comprising contacting the cell with the complex according to the invention, with a polynucleotide according to the invention, or with the recombinant expression vector according to the invention.

All the embodiments detailed in the preceding section in connection with the first aspect of the invention are also preferred embodiments according to this aspect of the invention.

In some embodiments, the in vitro methods of the invention comprise one step of contacting the cells, cell population or cell lines with the complex according to the invention, or with the polynucleotide according to the invention, or with the vector according to the invention.

Without being bound by any theory, the complex comprising the cell penetrating polypeptide (CPP) according to the invention may be endocytosed leading to conformational change of the translocated domain and the cargo molecule may finally be released in the cytoplasm from CPP.

The complex according to the invention, or the polynucleotide according to the invention, or the vector may have been purified before being used in the methods according to the invention.

The cell, cell population or cell line is preferably a eukaryotic cell or cell line. The cell, cell population or cell line may be an animal cell, cell population or cell line. The cell, cell population or cell line may be a mammalian cell, cell population or cell line. The cell, cell population or cell line may be a human cell, cell population or cell line. The cell, cell population or cell line may be a primary cell, cell population or cell line, in particular a human primary cell, cell population or cell line. The cell, cell population or cell line may be an immortalized cell, cell population or cell line, in particular a human immortalized cell, cell population or cell line. The cell, cell population or cell line may be an immune cell, cell population or cell line, in particular a human immune cell, cell population or cell line.

The cell, cell population or cell line may be a lymphocyte cell, cell population or cell line, in particular a B or T lymphocyte cell, cell population or cell line. The cell, cell population or cell line may be a neuron cell, cell population or cell line, in particular a primary neuron cell, or cell population. The cell, cell population or cell line may be an epithelial cell, cell population or cell line, in particular an epithelial cancer cell, or cell population.

The CPP according to the invention, the complex according to the invention, the fusion polypeptide according to the invention, the polynucleotide or set of polynucleotides according to the invention, or a vector according to the invention may for instance be used for the following applications:
gene editing (for instance using CRISPR/Cas 9), DNA fragment insertion into a genome,
gene expression modification (epigenetic modification such as methylation, mRNA degradation, for instance using CRISPR/Cas14a),
viral DNA clearing from a genome,
cell death induction of a specific cell type,
targeting of intracellular proteins or factors to the ubiquitin proteasome system and/or induction of intracellular protein degradation,
protein localization regulation,
CAR-T cell generation, and/or
enzyme replacement therapies.

A further object of the invention is a composition comprising a CPP according to the invention, a complex according to the invention, a fusion polypeptide according to the invention, a polynucleotide according to the invention, or a vector according to the invention.

All the embodiments detailed in the preceding section in connection with the first aspect of the invention are also preferred embodiments according to this aspect of the invention.

In some embodiment, the composition according to the present invention is a pharmaceutical composition or a medicament and further comprises at least one pharmaceutically acceptable excipient.

In some embodiment, the CPP according to the invention, the complex according to the invention, the fusion polypeptide according to the invention, the polynucleotide according to the invention, or the vector according to the invention, or the composition or pharmaceutical composition comprising the same, are formulated for administration to a subject.

In some embodiment, the CPP according to the invention, the complex according to the invention, the fusion polypeptide according to the invention, the polynucleotide according to the invention, or the vector according to the invention, or the composition or pharmaceutical composition comprising the same is/are to be administered systemically or locally.

In some embodiment, the CPP according to the invention, the complex according to the invention, the fusion polypeptide according to the invention, the polynucleotide according to the invention, or the vector according to the invention, or the composition or pharmaceutical composition comprising the same is/are to be administered by injection, orally, topically, nasally, buccally, rectally, vaginally, intratracheally, by endoscopy, transmucosally, or by percutaneous administration.

In some embodiment, the CPP according to the invention, the complex according to the invention, the fusion polypeptide according to the invention, the polynucleotide according to the invention, or the vector according to the invention, or the composition or pharmaceutical composition comprising the same is/are to be injected, preferably systemically injected.

Examples of formulations adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Examples of systemic injections include, but are not limited to, intravenous (iv) injection, subcutaneous injection, intramuscular (im) injection, intradermal (id) injection, intraperitoneal (ip), intranasal (in) injection and perfusion.

It will be understood that other suitable routes of administration are also contemplated in the present invention, and the administration mode will ultimately be decided by the attending physician within the scope of sound medical judgment.

In some embodiment, the CPP according to the invention, the complex according to the invention, the fusion polypeptide according to the invention, the polynucleotide according to the invention, or the vector according to the invention, or the composition or pharmaceutical composition comprising the same is/are to be administered to the subject in need thereof in a therapeutically effective amount.

It will be however understood that the dosage of the CPP according to the invention, the complex according to the invention, the fusion polypeptide according to the invention, the polynucleotide according to the invention, or the vector according to the invention, or the composition or pharmaceutical composition comprising the same will be decided by the attending physician within the scope of sound medical judgment. The specific therapeutically effective dose for any particular patient will depend upon a variety of factors including the disease being prevented and the severity of the disease; the activity of the CPP according to the invention, the complex according to the invention, the fusion polypeptide according to the invention, the polynucleotide according to the invention, or the vector according to the invention, or the composition or pharmaceutical composition comprising the same employed; the age, body weight, general health, sex and diet of the subject; the time of administration, route of administration, and rate of excretion of the CPP according to the invention, the complex according to the invention, the fusion polypeptide according to the invention, the polynucleotide according to the invention, or the vector according to the invention, or the composition or pharmaceutical composition comprising the same employed; the duration and regimen of the treatment; drugs used in combination or coincidental with the CPP according to the invention, the complex according to the invention, the fusion polypeptide according to the invention, the polynucleotide according to the invention, or the vector according to the invention, or the composition or pharmaceutical composition comprising the same employed; and like factors well known in the medical arts.

In some embodiments, the CPP according to the invention, the complex according to the invention, the fusion polypeptide according to the invention, the polynucleotide according to the invention, or the vector according to the invention, or the composition or pharmaceutical composition comprising the same are for use as a medicament, in particular, for use in the prevention and/or treatment of a disease.

The disease to be prevented and/or treated may for instance be an α-synucleinopathy such as e.g. Parkinson's disease, multiple system atrophy, dementia with Lewy bodies; a tauopathy such as e.g. Alzheimer's disease, front temporal dementia, Pick's disease; or a tumor or cancer.

Therefore, in some embodiments, the CPP according to the invention, the complex according to the invention, the fusion polypeptide according to the invention, the polynucleotide according to the invention, or the vector according to the invention, or the composition or pharmaceutical composition comprising the same are for use as a medicament.

In some embodiments, the CPP according to the invention, the complex according to the invention, the fusion polypeptide according to the invention, the polynucleotide according to the invention, or the vector according to the invention, or the composition or pharmaceutical composition comprising the same are for use in the prevention and/or treatment of an α-synucleinopathy such as e.g. Parkinson's disease, multiple system atrophy, dementia with Lewy bodies; a tauopathy such as e.g. Alzheimer's disease, front temporal dementia, Pick's disease; a tumor or cancer.

The CPP according to the invention, the complex according to the invention, the fusion polypeptide according to the invention, the polynucleotide according to the invention, or the vector according to the invention, or the composition or pharmaceutical composition comprising the same may also be for use in cell therapies. For instance, it may be used for CAR-T cell generation, or it may be for use in allogenic CAR-T cell therapy, or allogenic stem cell therapy, or enzyme replacement therapy.

In some embodiment, the method according to the invention is used to prevent and/or treat a disease in a subject in need thereof, comprising administering to the subject CPP according to the invention, the complex according to the invention, the fusion polypeptide according to the invention, the polynucleotide according to the invention, or the vector according to the invention, or the composition or pharmaceutical composition comprising the same.

In some embodiment, a therapeutically effective amount of the at least one CPP according to the invention, the complex according to the invention, the fusion polypeptide according to the invention, the polynucleotide according to the invention, or the vector according to the invention, or the composition or pharmaceutical composition comprising the same is administered at least once a day, twice a day, at least three times a day or at least four times a day.

In other embodiments, a therapeutically effective amount of the at least one CPP according to the invention, the complex according to the invention, the fusion polypeptide according to the invention, the polynucleotide according to the invention, or the vector according to the invention, or the composition or pharmaceutical composition comprising the same is administered every two, three, four, five, or six days.

In other embodiments, a therapeutically effective amount of the at least one CPP according to the invention, the complex according to the invention, the fusion polypeptide according to the invention, the polynucleotide according to the invention, or the vector according to the invention, or the composition or pharmaceutical composition comprising the same is administered twice a week, every week, every two weeks, or once a month.

In other embodiments, a therapeutically effective amount of the at least one CPP according to the invention, the complex according to the invention, the fusion polypeptide according to the invention, the polynucleotide according to the invention, or the vector according to the invention, or the composition or pharmaceutical composition comprising the same is administered every month for a period at least 2; 3; 4; 5; 6 months or for the rest of the life of the subject.

In other embodiments, a therapeutically effective amount of the at least one CPP according to the invention, the complex according to the invention, the fusion polypeptide according to the invention, the polynucleotide according to the invention, or the vector according to the invention, or the composition or pharmaceutical composition comprising the same ranges from about 1 μg to 100 g, 1 mg to 1 g, 10 mg to 500 mg.

In other embodiments, a therapeutically effective amount of the at least one CPP according to the invention, the complex according to the invention, the fusion polypeptide according to the invention, the polynucleotide according to the invention, or the vector according to the invention, or the composition or pharmaceutical composition comprising the same ranges from about 10 to 100 mg, preferably 60 mg.

In other embodiments, a therapeutically effective amount of the at least CPP according to the invention, the complex according to the invention, the fusion polypeptide according to the invention, the polynucleotide according to the invention, or the vector according to the invention, or the composition or pharmaceutical composition comprising the same ranges from about 0.1 μg/kg to 1 g/kg of body weight, 0.1 mg/kg of body weight to 500 mg/kg, 10 mg/kg to 100 mg/kg of body weight.

The present invention also relates to an in vitro assay for the incorporation of a molecule of interest into a cell or cell population comprising the steps of:
obtaining a purified complex comprising a cell penetrating polypeptide (CPP) according to the invention associated to a molecule of interest;
contacting said complex with a cell or cell population; thereby incorporating the molecule of interest in the cell or cell population.

In some embodiment, the in vitro assay is for incorporation of an antibody in a cell or a cell population.

In some embodiment, the assay is for incorporation of a complex or complex of molecules in a cell or cell population.

In some embodiment, the in vitro assay is for modifying the DNA material in a cell or cell population by incorporation of a CRISPR associated protein in complex with a sgRNA.

### BRIEF DESCRIPTION OF THE DRAWINGS

FIG. 1 is a schematic representation of the intracellular delivery strategy using the CNF1 delivery mechanism by replacing the Catalytic domain (C domain) with a cargo of interest. 1) The A1 & A2 domains of CNF1 allow receptor binding. 2) CNF1 enters cells via endocytosis. 3) Upon endosome acidification, conformation changes of CNF1 occur, releasing thereby the cargo through the plasma membrane and into the cytosol of the host cell.

FIG. 2 shows the plasmid vector for the expression of FlagSE-CRISPR Cas9EGFP.

FIG. 3 shows a gene knock out using the SE-CRISPR Cas9EGFP. a) SE-CRISPR/Cas9EGFP successfully knocks out gene expression. HeLa Cells stably expressing EGFP-p65 were treated with either control or purified FlagSE-CRISPR/Cas9EGFP. Cells were fixed 20 hours post treatment, permeabilized and the nuclei was stained using DAPI. The coverslips were analyzed using a fluorescence microscope. ImageJ analysis of the pictures taken are presented in FIG. 3a). The box and whiskers plot represent the EGFP intensity of untreated vs FlagSE-CRISPR/Cas9EGFP treated cells. b) SE-CRISPR/Cas9EGFP successfully knocks out EGFP gene expression. HeLa Cells stably expressing EGFP-p65 were treated with either control or purified FlagSE-CRISPR/Cas9EGFP. Cells were lysed 20 hours post treatment using 2× laemmli buffer. The proteins of each condition were separated using an SDS/Page before being transferred to a PVDF membrane. The Knock-out efficacy was revealed using an antibody against EGFP and the loading control with an antibody against GAPDH. Immunoblot Quantification shows the decrease in EGFP protein levels upon FlagSE-CRISPR/Cas9EGFP treatment.

FIG. 4 shows the generation and test of the SE-NanoEGFP plasmid. a) Plasmid map for the expression of OCDM-Nanobody against EGFP (FlagSE-NANOEGFP). b) Immunofluorescence showing colocalization of cytosolic F1agSE-NanoEGFP with EGFP-Rab6. Hek293T cells seeded on coverslips were transfected using EGFP-Rab6 plasmid for 24 hours. The cells were then treated with 5% of purified F1agSE-NanoEGFP for 1 hour. The cells were washed twice using PBS before being fixed, permeabilized and cytosolic FlagSE-NanoEGFP was stained using FLAGM2 antibody that was revealed using a Texas red coupled secondary antibody against mouse IgG.

FIG. 5 shows the in cellulo knock-out of TCR alpha in primary T cells from a healthy donor. a) Primary CD4+ T cells from a healthy donor were activated using CD3/CD28 beads and complemented with IL2 (10 ng/mL) as described in the methods. The cells exhibited very strong proliferation after day 3 of stimulation. b) Number of activated CD4+ T cells 24 hours and 48 hours after treatment with 5 μg of FLAGSE (condition 1), FLAGSE-CRISPR/Cas9_{TCRα1} (condition 2) and FLAGSE-CRISPR/Cas9_{TCRα2} (condition 3). c) Immunoblot quantification showing the decrease in TCRα protein levels in whole lysates of cells treated for 48 hours as in 5b. Cells were lysed using 2× Laemmli buffer with a reducing agent. The proteins of each condition were separated using an SDS/PAGE before being transferred to a PVDF membrane. The knock-out efficacy was revealed using an antibody against TCRα (Catalog #TCR1145) and the protein loading consistency was verified with an antibody against GAPDH. Fold change to condition 1. d) Geometric mean of TCRα fluorescence, measured in a flow cytometry assay using Flowjo software (in arbitrary units) and showing a decrease in TCRα levels from cells in condition 2 and 3 compared to condition 1 (treatment as in b). Fixed cells were treated for 1 hour with TCRα primary antibody (diluted to 1 to 50 in PBS with 0.1% TWEEN 20). The cells were washed twice with PBS before staining with a secondary antibody couple to FITC (diluted 1 to 1000 in PBS with 0.1% TWEEN 20) for 1 hour before analysis using a CytoFLEX from Beckman Coulter.

FIG. 6 shows in cellulo knock-out of SNCAm in primary neurons. a) Overview of the procedure for in vitro SNCA KO in primary neurons. b) Picture of neuronal striatum cells on Day 2 and Day 10 respectively. c) Immunoblot quantification showing a strong decrease in SNCA protein levels in whole lysates of cells treated for 48 hours with FlagSE-CRISPR/CassNCAm compared to control. Cells were lysed using 2× laemmli buffer with a reducing agent. The proteins of each condition were separated using an SDS/Page before being transferred to a PVDF membrane. The knock-out efficacy was revealed using an antibody against SNCA (Catalog #PA5-85791) and the loading control with an antibody against GAPDH.

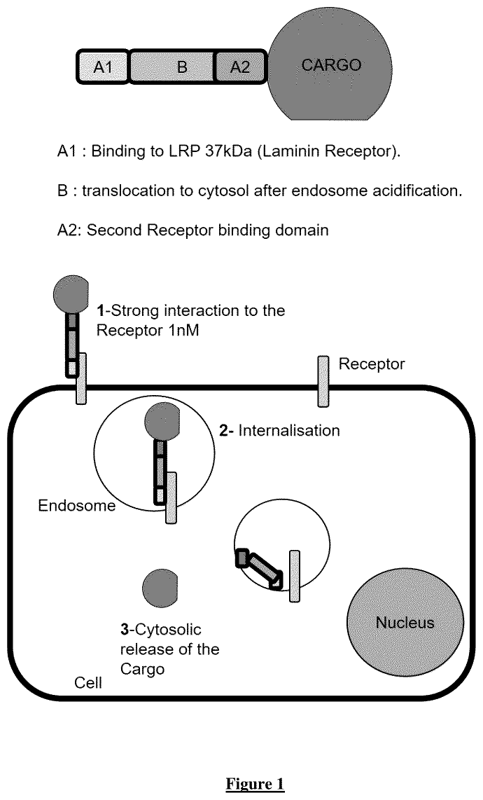

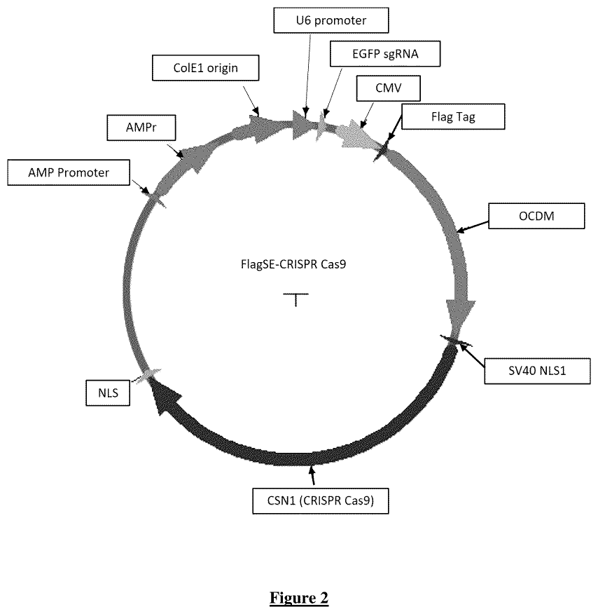

### EXAMPLES

The present invention is further illustrated by the following examples.

### Example 1: Delivery of a Large Functional Ribonucleoprotein

### Materials and Methods

### Creation of an Expression Vector Containing a Recombinant Protein.

The DNA sequence coding for the delivery mechanism of Cytotoxic Necrotizing Factor 1 (CNF1) was generated using gene art services. This sequence will be referred to as OCDM an acronym for Optimized sequence of CNF Delivery Mechanism. This sequence is optimized for expression in mammalian cells. The OCDM was amplified by PCR using the following probes (1FWD: ACGACAAGCTTGCGGCCGCGAATTCACATCACCATCACCAT (SEQ ID NO: 30) 1RVS: GATGCCACCCGGGATCCTCTCCAGGCCGATGCTGTACTTCT (SEQ ID NO: 31)). 1 μg of the PCR product was digested using EcoR1 (R0101S) and XmaI (R0180S) following manufacturer's protocol. 100 ng of digested OCDM was ligated into 50 ng of pre-linearized pCMV-Flag vector with EcoR1 and XmaI. The CMV-flag-OCDM sequence was then amplified using the following probes (2FWD: CAAATGGCTCTAGAGGTACCCGTTACATAACTTACGGTAA (SEQ ID NO: 32) 2RVS: CCGATGCTGTACTTCTTGTCG (SEQ ID NO: 33)) and the pSpCas9(BB)-2APuro (Addgene 62988 Feng Zhang Lab) was amplified using the following probes (3FWD: GACAAGAAGTACAGCATCGGC (SEQ ID NO: 34) 3RVS: GGTACCTCTAGAGCCATTTG (SEQ ID NO: 35). 100 ng of each PCR product was mixed and stitched using the Geneart GIBSON mastermix (HiFi A46628) following manufacturer's protocol. The final assembled vector as illustrated in the figure (FIG. 2) contains thereby the U6 promoter followed by the sequence encoding single guide RNA structure against EGFP (guide FWD: GGGCGAGGAGCTGTTCACCG (SEQ ID NO: 36); guide RVS: AAACCGGTGAACAGCTCCTCGCCC (SEQ ID NO: 37)) and the OCDM sequence upstream of csn1 gene (CRISPR Cas9) from S. pyrogenes under the control of the CMV promoter. Clustered Regularly Interspaced Short Palindromic Repeats (CRISPR) CRISPR associated protein (Cas9), binds to a synthetic single guide RNA (sgRNA) to form a Ribonucleotide complex that can break Hydrogen bonds of double stranded DNA. The sgRNA will guide CRISPR Cas9 to the complementary 20 nucleotides sequence upstream of a Protospacer Adjacent Motif (PAM) sequence. This construct will allow therefore to express the synthetic fusion of the OCDM with CRISPR CAS9, along with the single guide RNA against EGFP to assemble a functional ribonucleoprotein complex.

### Recombinant Protein Expression in HEK293T Cells.

Cell expression protocol: 4·10⁶ HEK293T Cells were seeded in a 100 mm2 cell plate. The next day, cells were transfected with 5 μg of the plasmid using lipofectamine 2000 (11668027 from ThermoFisher Scientist) and following manufacturer's protocol. 48 hours post-transfection cells were lysed with multiple buffers and methods to test solubility and extraction.

The optimal lysis protocol was determined as follows: Plated cells were put on ice and “washed with pre-chilled PBS to remove remaining cell medium. 150 μl of lysis buffer (Tris-HCl 50 mM pH8, NaCl 100 mM, pmsf 100 nM) was added to the plates and incubated for 10 min on ice. The cells were scraped and transferred to an Eppendorf tube, before soft sonication (5 times 15 seconds pulses at 15%). Cells were centrifuged at 10,000 g for 10 min and the soluble fraction was then transferred to a clean tube and the pellet discarded. 30 μl of the soluble fraction was mixed with 10 μl of loading buffer (Laemmli 4× supplemented with DTT at 400 mM). The lysates were casted on a 4-12% Tris-Glycine Gel (Thermo NP0322BOX) and transferred to a PVDF membrane. The expression levels of the synthetic construct were assessed by Western blot using a FLAG-M2 primary antibody (sigma F1804) and secondary anti-mouse HRP (data not shown).

### Ribonucleoproteic Complex Purification

3×100 mm2 dishes were seeded with 5*10⁶ HEK293T cells. After 24 hours, the cells were transfected with 5 μg of the plasmid using lipofectamine 2000 and following manufacturer's protocol. 48 hours post transfection, each plate of cells was lysed with 500 μl of the optimal lysis buffer and following protocol. Lysates were pooled into a single 2 ml tube. 40 μl of the total cleared lysate (2.5%) was taken and the rest was incubated with 100 μl of Slurry FlagM2-beads (ref) for 2 hours. The beads were centrifuged at 500 g for 2 min and washed with 1.5 ml of lysis buffer 4 times. The recombinant protein was eluted by competition using 200 μl of Flag peptide for 30 min at 4 C (Flag [0.5 mg/ml] in Tris-HCl 50 mM pH8, NaCl 100 mM, MgCl2 10 mM). Loading buffer was added to the beads to assess elution efficacy. Input, beads and elution fractions were resolved by Western Blot to assess purification efficacy (data not shown). 30 μl of the purification were incubated with 300 ng of EGFP plasmid diluted in the elution buffer and vector linearization was assessed by DNA electrophoresis using 1% agarose gel in TAE buffer (data not shown).

### In Cellulo Knock Out of EGFP

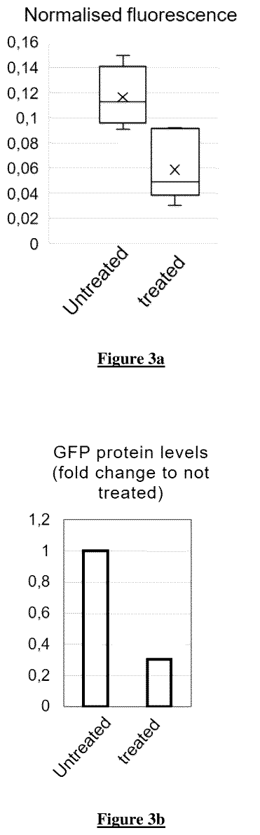

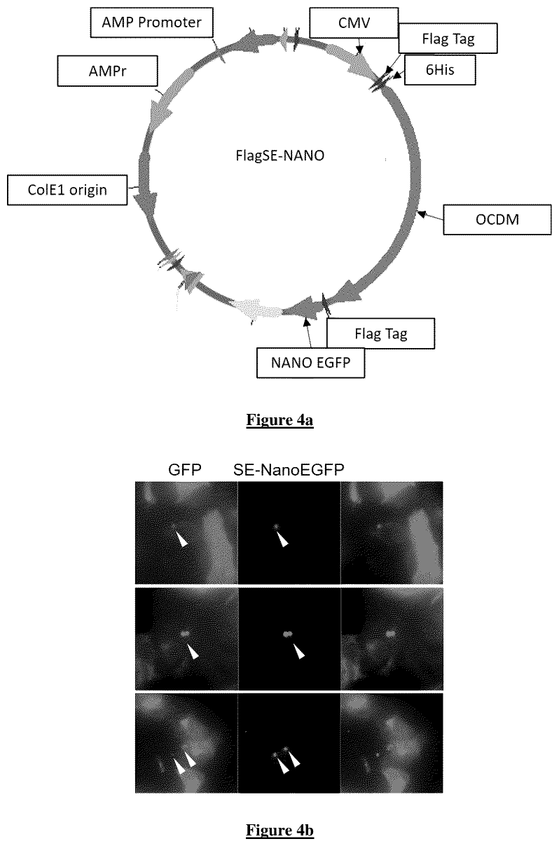

25,000 HeLa Cells stably expressing EGFP-p65 were seeded on p24 with coverslips and were treated with either 40 μl of the elution buffer or 40 μl of recombinant OCDM-CRISPR Cas9 protein that we will thereafter call FlagSE-CRISPR Cas9EGFP (SE stands for Self-Entering, EGFP stands for the single RNA guide targeting EGFP). 24 hours post treatment cells were fixed with 4% paraformaldehyde in PBS and the coverslips were mounted using MOWIOL (Merck 81381) supplemented with Hoescht (1/50 000). The mean fluorescence was assessed using a fluorescence microscope (Olympus BX53F2) and quantified using ImageJ (FIG. 3a). 25 000 HeLa Cells stably expressing EGFP-p65 were seeded on p48 wells before being treated with either 40 μl of the elution buffer or 40 μl of recombinant FlagSE-CRISPR Cas9EGFP. 24 hours post treatment cells were lysed using Laemmli buffer supplemented with DTT 100 mM. Lysates were resolved on a 4-12% Tris-Glycine acrylamide gel (Thermo NP0322BOX) before transfer to a PVDF membrane. The levels of EGFP were revealed by Immunoblotting using the primary antibody (Novus NB600-308), and secondary anti-rabbit antibody coupled to HRP (FIG. 3b).

### Results

The experimental results of example 1 determined that the CNF1 entry mechanism can be grafted to a very large ribonucleoprotein called CRISPR Cas9 and an associated sgRNA. The final synthetic protein is soluble and can be purified using affinity chromatography). Moreover, the co-expression of the single guide RNA with the FlagSE-CRISPR Cas9 allowed the purification of a functional ready to use complex that can cut DNA in vitro. Treating cells stably expressing EGFP-p65 with FlagSE-CRISPR Cas9EGFP turns off EGFP expression in these cells as measured by fluorescence or by western blot in. Overall, the results from example 1 show that the delivery mechanism of CNF1 can be used to deliver a functional large ribonucleoprotein into cells.

### Example 2: Delivery of a Small Single Chain Antibody

### Materials and Methods

### Creation of an Expression Vector Containing a Recombinant Protein.

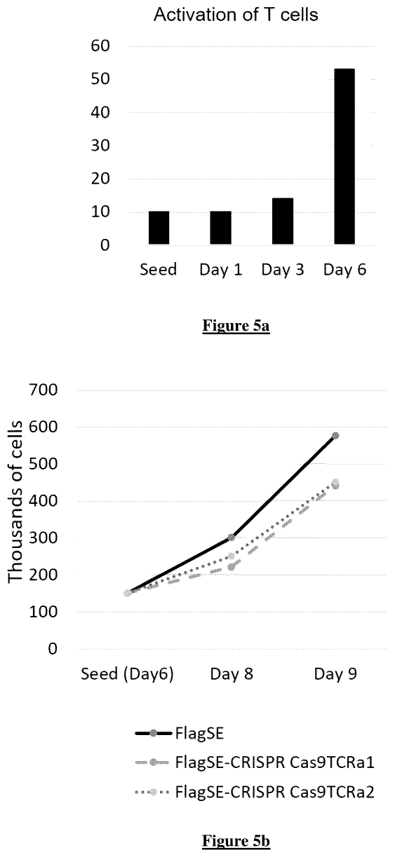

The OCDM was amplified by PCR using the following probes (4FWD: CTTGTCGTCATCGTCTTTGTAGTCGTCAACTTCGTC (SEQ ID NO: 38) 4RVS: GCCGCGAATTCACATCACCATCACCATCAC (SEQ ID NO: 39)). The pCMV-Flag vector was then amplified using the following probes (5FWD: GTGATGGTGATGGTGATGTGAATTCGCGGCCGCAAG (SEQ ID NO: 40) 5RVS: acccaggttaccgttagcagcAGAGGATCCCGGGTGGCAT (SEQ ID NO: 41)) and the sequence of the single chain antibody, here called nanobody, targeting EGFP was amplified using the following probes (6FWD: GACTACAAAGACGATGACGACAAGGTTCAGCTGGTTGAA (SEQ ID NO: 42) 6RVS: GATGCCACCCGGGATCCTCTGCTGCTAACGGTAAC (SEQ ID NO: 43)) from the plasmid (pGEX6P1-GFP-Nanobody) (Addgene 61838). 100 pmol of each PCR product was mixed and stitched using the geneart GIBSON mastermix and following manufacturer's protocol. The final assembled vector as illustrated in the FIG. 4a) contains thereby the OCDM sequence followed by the EGFP targeting nanobody under the control of a CMV promoter.

### Expression and Purification of Nanobodies in HEK293T Cells

Two p12 wells were seeded with 10⁵ HEK293T cells. The next day, cells were transfected with 0.5 μg of the plasmid using lipofectamine 2000 and following manufacturer's protocol. Cells were lysed as follows: Plated cells were put on ice and “washed with pre-chilled PBS to remove remaining cell medium. 100 μl of lysis buffer (Tris-HCl 50 mM pH8, NaCl 100 mM, pmsf 100 nM) was added to the plates and incubated for 10 min on ice. Cell lysates were centrifuged at 10 000 g for 10 min, the soluble fraction was then transferred to a clean tube (1.5 mL) and the pellet discarded. 30 μl of the soluble fraction was mixed with 10 μl of loading buffer (Laemmli 4× supplemented with DTT at 400 mM). The lysates were casted on a 4-12% Tris-Glycine Gel (Thermo NP0322BOX) and transferred to a PVDF membrane. The expression levels of the synthetic construct were assessed by Western blot using a Flag-M2 primary antibody (sigma F1804) and secondary anti-mouse HRP (data not shown).

Five T75 flasks were seeded with 5*10⁶ cells of HEK293T cells. The next day the cells were transfected each with 5 μg of the expression vector FlagSE-NanoEGFP using lipofectamine 2000 and following manufacturer's protocol. 24 h post transfection each of the plates were lysed with 500 μl of lysis buffer at 4° C. before being pooled into a single tube. The total lysates were syringed and centrifuged at 10 000 g for 10 min. The soluble fraction was transferred into a 15 ml tube, and the pellet was discarded. 400 μl of slurry FlagM2 resin was equilibrated using 1 ml of lysis buffer and mixed with the total lysate for 2 hours at 4° C. to purify the recombinant proteins. The beads were then centrifuged at 500 g for 2 min and washed with 1.5 ml of lysis buffer 4 times. The recombinant protein was eluted by competition using 1 ml of Flag peptide for 30 min at 4° C. (Flag [0.1 mg/ml] in Tris-HCl 50 mM pH8, NaCl 100 mM, MgCl2 10 mM). 50 μl of the elution fraction were resolved on a Tris-Glycine 4-12% gel and coloured using Instant Blue (ab119211) to assess purification efficacy and purity (data not shown).

### HEK293T Cytosolic Delivery of Nanobodies

50 000 HEK293T cells were seeded on three p12 wells with coverslips. The following day each well was transfected using lipofectamine 2000 with 500 ng of EGFP-Rab6 expression vector. The next day two of the p12 wells were treated with 50 μl of the purified batch and one well was treated with 50 μl of the elution buffer. One-hour post treatment cells were fixed with 4% paraformaldehyde in PBS, before permeabilization using Triton at 0.1% for 10 min. The wells were then incubated with 500 μl of PBS supplemented with 5% BSA for one hour to block nonspecific antibody binding. The coverslips were then stained with a FlagM2 primary antibody solution (dilution to a factor of 500 in PBS-BSA 5%) followed by secondary antibody staining against mouse IGG conjugated with Alexa 594. The coverslips were mounted using MOWIOL supplemented with Hoescht (1/50 000). The coverslips were then analysed using a Fluorescence microscope (FIG. 4b).

### Results

The experimental results of example 2 determined that the CNF1 entry mechanism can be grafted to a small single chain antibody against EGFP (hereafter named FlagSE-NanoEGFP). The final synthetic protein is soluble and can be purified using affinity chromatography. Finally, cells expressing EGFP-Rab6 were treated with the FlagSE-NanoEGFP for one hour. F1agSE-NanoEGFP was stained using Flag antibodies and the cytosolic localization of the synthetic nanobodies with EGFP-Rab6 was observed. Overall, the results from example 2 confirm that the delivery mechanism of CNF1 can be used to deliver small single chain antibodies.

### Example 3: CNF as an Exogenous Carrier to Edit Primary Immune Cells Using CRISPR/Cas Systems

### Materials and Methods

Insertion of sgRNA Guides Against the TCR Alpha Subunit Gene in the Expression Vector for FLAGSE-CRISPR/Cas9.

Two independent guides against TCR alpha constant region were inserted in a custom vector containing the U6 promoter and the OCDM-CRISPR/Cas9 as described in Example 1.

TCRα guide1 FWD: GATTAAACCCGGCCACTTTCAGG (SEQ ID NO: 48).

TCRα guide1 RVS: CCTGAAAGTGGCCGGGTTTAATC (SEQ ID NO: 49).

TCRα guide2 FWD: TGTGCTAGACATGAGGTCTA (SEQ ID NO: 50).

TCRα guide2 RVS: TAGACCTCATGTCTAGCACA (SEQ ID NO: 51).

Two independent vectors named FLAGSE-CRISPR/Cas9_{TCRα1} and FLAGSE-CRISPR/Cas9_{TCRα2} were therefore obtained. These constructs allow the expression the synthetic fusion product of the OCDM with CRISPR/Cas9, along with two single guide RNAs against TCRα to assemble a functional complex.

FLAGSE-CRISPR/Cas9_{TCRα1} and FLAGSE-CRISPR/Cas9_{TCRα2} Complex Purification

Five T175 flasks were seeded with 10⁷ HEK293T cells. After 24 hours, the cells of each flask were transfected with 5 μg of the plasmid using lipofectamine 2000 following the manufacturer's protocol. 48 hours post transfection, the cells were detached using Trypsin-EDTA. Cells were then centrifuged at 500 g and washed once with PBS before lysisng with 20 mL of the optimal lysis buffer using the same protocol as shown in Example 1. After sonication the insoluble fraction was cleared from total lysates via centrifugation (10 000 g for 10 min). The insoluble fraction was discarded, and the soluble fraction was incubated with 400 μL of equilibrated Slurry FLAGM2-beads for 2 hours to trap the soluble Flag tagged proteins. The beads were centrifuged at 500×g for 2 min and washed with 5 mL of lysis buffer 4 times. The recombinant protein was eluted by competition using 500 μL of FLAG peptide for 1 hour at 4° C. (FLAG [0.5 mg/mL] in Tris-HCl 50 mM pH 8, NaCl 100 mM, MgCl2 10 mM). 50 μL of the elution fraction were separated on a Tris-Glycine 4-12% gel and dyed using Instant Blue (ab119211) to assess purification efficacy and purity (data not shown).

In Cellulo Knock Out of TCR Alpha in Primary T Cells from a Healthy Donor.

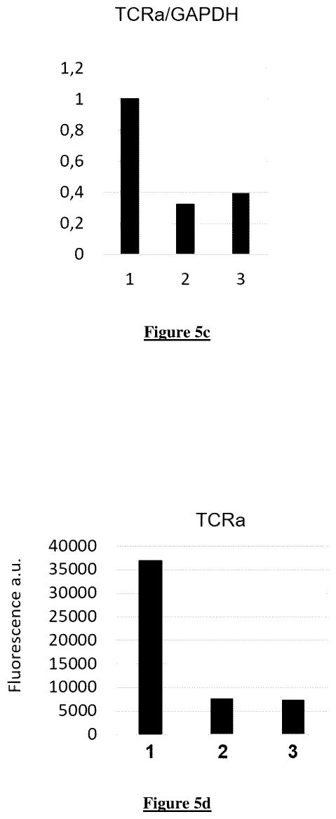

A vial of 10⁷ million T Lymphocytes (acquired from Lonza donor number: 42819), was thawed in complete medium (RPMI supplemented with Glutamine and 10% of heat inactivated Human AB Serum). The following day, the cells were activated with 10⁷ CD3/CD28 beads and IL-2 (10 ng/mL) (the cells were fed by exchanging the media every 2 days). The cells were counted every day and checked for viability using Trypan Blue. T Cell activation and proliferation was achieved after 3 days (FIG. 5a).

On Day 7, 1.5×10⁶ activated T cells were equally distributed in a 12 well plate. The next day cells were treated with approximately 5 μg of either control (FLAGSE—condition 1), FLAGSE-CRISPR/Cas9_{TCRα1} (condition 2) and FLAGSE-CRISPR/Cas9_{TCRα2} (condition 3). 48 h after treatment the cells were counted and checked for viability using trypan blue (less than 1% in all conditions) (FIG. 5b). The cells were either lysed using Laemmli buffer to visualize total TCRα protein levels by immunoblot (FIG. 5c) or fixed with 4% PFA for cytometry analysis (FIG. 5d).

### Results:

FLAGSE-CRISPR/Cas9_{TCRα1} and FLAGSE-CRISPR/Cas9_{TCRα2} exhibited very high efficiency in knocking out the expression of the TCR alpha subunit of the TCR complex as measured by Western blot and flow cytometry. Cells treated with either control FLAGSE (the delivery domain), FLAGSE-CRISPR/Cas9_{TCRα1}, or FLAGSE-CRISPR/Cas9_{TCRα2} did not exhibit cell toxicity or cell death, and continued to divide 48 hours after treatment. Thereby FLAGSE-CRISPR/Cas9 can be used to generate allogenic immune cells (such as T lymphocytes lacking the TCR) or used for the insertion of a CAR receptor into immune cells (such as CAR-T cells).

### Example 4: CNF as an Exogenous Carrier to Edit Neurons Using the CRISPR/Cas System

### Materials and Methods

Insertion of sgRNA Guides Against the Mouse Alpha Synuclein (SNCAm) Gene in the Expression Vector for FlagSE-CRISPR/Cas9.

The DNA sequence coding for RNA guide against SNCAm was inserted in the custom vector containing the U6 promoter and the OCDM-CRISPR/Cas9 as described in Example 1.

SNCAₘ guide1 FWD:
(SEQ ID NO: 52)
AGGGAGTCCTCTATGTAGGTAGG
SNCAₘ guide1 RVS:
(SEQ ID NO: 53)
CCTACCTACATAGAGGACTCCCT

The vector named FlagSE-CRISPR/Cas9NCAm was thus obtained, allowing the expression of the synthetic fusion of the OCDM with CRISPR/CAS9, along with the single guide RNA against SNCA. to assemble a functional complex.

### FlagSE-CRISPR/Cas9_{SNCAm} Purification

Five T175 flasks were seeded with 10⁷ HEK293T cells. After 24 hours, the cells of each flask were transfected with 5 μg of the plasmid using lipofectamine 2000 and following manufacturer's protocol. 48 hours post transfection, the cells were detached using Trypsin-EDTA. Cells were then centrifuged at 500 g and washed once with PBS before lysis with 20 ml of the optimal lysis buffer and following the same protocol as in example 1. After sonication the total lysates were cleared via centrifugation (10 000 g×10 min). The insoluble fraction was discarded, and the rest was incubated with 400 μl of equilibrated Slurry FlagM2-beads for 2 hours to trap soluble flag tagged proteins. The beads were centrifuged at 500 g for 2 min and washed with 5 ml of lysis buffer 4 times. The recombinant protein was eluted by competition using 500 μl of Flag peptide for 1 hour at 4° C. (Flag [0.5 mg/ml] in Tris-HCl 50 mM pH8, NaCl 100 mM, MgCl2 10 mM). 50 μl of the elution fraction were resolved on a Tris-Glycine 4-12% gel and colored using Instant Blue (ab119211) to assess purification efficacy and purity (data not shown).

### In Cellulo Knock Out of SNCAm in Primary Neurons.

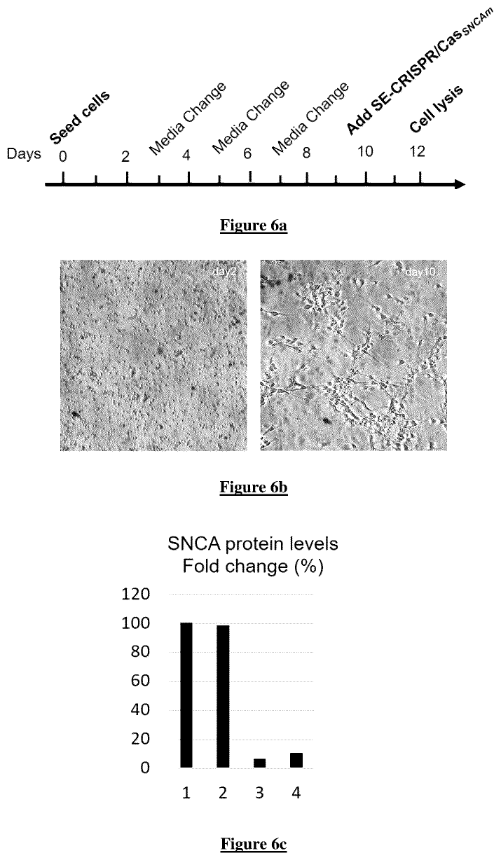

A vial of 4 million primary mouse brain striatum neuronal cells (acquired from Lonza ref: M-CP-402), was thawed in the defined medium as recommended by Lonza (PNGM™ BulletKit™) in 4 p12 wells pre-coated overnight with Laminin (30 μg/ml) and (poly-D-lysin 50 μg/ml). 50% of the media was changed every 3 days. Neurite formation was observed a week after thawing (FIG. 6b).

On Day 10, cells were treated with approximately 5 μg of control (FlagSE—condition 1), 10 μg of control (FlagSE—condition 2), 5 μg of FlagSE-CRISPR/Cas9NCAm (condition 3) or 10 μg of FlagSE-CRISPR/Cas9sNCAm (condition 4) (FIG. 6a). 48 h after treatment cells were lysed using laemmli to control total SNCA protein levels by immunoblot. GAPDH was used as a loading control (FIG. 6c).

### Results:

FlagSE-CRISPR/Cas9sNCAm exhibits very high efficiency in knocking out the expression of alpha synuclein as measured by Western blot. Treated cells with either control FlagSE (the delivery domain) or FlagSE-CRISPR/Cas9sNcAm did not exhibit cell toxicity 48 hours after treatment. Thereby, FlagSE-CRISPR/Cas9 can be used to edit genes in neurons for applications in neurobiology. Blocking the expression of alpha-synuclein using FlagSE-CRISPR/Cas9 may thus be a therapeutical solution for the treatment of Parkinson's.

Part of the Sequence listing

| SEQ | | |
|---|---|---|
| ID | | |
| NO | Name | Sequence 5′-3′ |
| 9 | CNF1 | MGNQWQQKYLLEYNELVSNFPSPERVVSDYIKNCFKTDLPWFSRIDPDNAYFICFSQNRSNSRSYTGWDHLGKYKTE |
| | LRP37kDa | VLTLTQAALINIGYRFDVFDDANSSTGIYKTKSADVFNEENEEKMLPSEYLHFLQKCDFAGVYGKTLSDYWSKYYDK |
| | domain | FKLLLKNYYISSALYLYKNGELDEREYNFSMNALNRSDNISLLFFDIYGYYASDIFVAKNNDKVMLFIP |
| 10 | CNF1 | GAKKPFLFKKNIADLRLTLKELIKDSDKQQLLSQHFSLYSRQDGVSYAGVNSVLHAIENDGNFNESYFLYSNKTLSNK |
| | trans- | DVFDAIAISVKKRSFSDGDIVIKSNSEAQRDYALTILQTILSMTPIFDIVVPEVSVPLGLGIITSSMGISFDQLINGDTYEER |
| | location | RSAIPGLATNAVLLGLSFAIPLLISKAGINQEVLSSVINNEGRTLNETNIDIFLKEYGIAEDSISSTNLLDVKLKSSGQHVN |
| | domain | IVKLSDEDNQIVAVKGSSLSGIYYEVDIETGYEILSRRIYRTEYNNEILWTRGGGLKGGQPFDFESLNIPVFFKDEPYSAV |
| | | TGSPLSFINDDSSLLYPDTNPKLPQPTSEMDIVNYVKGSGSFGDRFVTLMRGATEEEAWNIASYHTAGGSTEELHEILL |
| | | GQGPQSSLGFTEYTSNVNSADAASRRHFLVVIKVHVKYITNNNVSYVNHWAIPDEAPVEVLA |
| 11 | CNF1 | VVDRRFNFPEPSTPPDISTIRKLLSLRYFKESIESTSKSNFQKLSRG |
| | Lu/BCAM | |
| | domain | |
| 12 | CNF2 | MNVQWQQKYLLEYNELVSNFPSPERVVSDYIRRCFKTDLPWFSQVDPDNTYFIRFSQSRSNSRSYTGWDHLGKYKTG |
| | LRP37kDa | VLTLTQAALINIGYHFDVFDDANASAGIYKTSSADMFNEKNEEKMLPSEYLYFLKGCDFSGIYGRFLSDYWSKYYDKF |
| | domain | KLLLKNYYISSALYLYKNGEIDEYEYNFSISALNRRDNISLFFFDIYGYYSSDMFVAKNNERVMLFIP |
| 13 | CNF2 | GAKKPFLFEKNIADLRISLKNLIKENDNKQLLSQHFSLYSRQDGITYAGVNSVLNAIENDGVFNESYFLYSNKRINNKD |
| | trans- | VFDAVAFSVKKRSFSDGDIVIKSNSEAQRDYALTILQTILSMTPIFDVAIPEVSVTLGLGIIASSMGISFDQLINGDTYEER |
| | location | RSAIPGLATNAALLGLSFAIPFLISKAGTNQKILSRYTKHEIRTLNETNIDMFLEEYGINKNSISETKVLEVELKGSGQHV |
| | domain | NIVKLSDEDSKIVAVKGNSLSGIYYEVDIETGYEISSRRIYRTEYNDKIFWTRGGGLKGGQSFDFESLKLPIFFKDEPYSA |
| | | VPGSSLSFINDDSSLLYPNSTPKLPQPTPEMEIVNYVKRAGDFGERLVTLMRGTTEEEAWNIARYHTAGGSTEELHEIL |
| | | LGQGPQSSLGFTEYTSNINSADAASRRHFLVVIKVQVKYINNNNVSHVNHWAIPDEAPVEVLA |
| 14 | CNF2 | VVDRRFNFPEPSTPPNISIIHKLLSLRYFKENIESTSRLNLQKLNRG |
| | Lu/BCAM | |
| | domain | |
| 15 | CNF3 | MNTQWQQKYLLEYNDLVSKFPSPEKVTSDYIKHKFKTDLPWFSRVDPDKTYFIQFSQNRSNSRSYTGWDHLGKYKT |
| | LRP37kDa | DALTLTQAAIINIGYRFEVFDEANATAGIYTTNNADLFDETNEAKMLPSEYLYFLKNCDFAGLYNKALSDYWSKNHE |
| | domain | KFKLLLKNYYISSSLYLYKNNVISKDEHEFTMKALNRDDNIELFSFDIYGYYSSDIFGAKNDDRIMLFIP |
| 16 | CNF3 | GATNPFLFSENISHLRTHLKELIKENDNRELLSRHFSLYDCQDGSTFYGVDSVLKEIVNGNFNESYFMYTYKKFNERDV |
| | trans- | FDAISFSVQKRSFSDGDTIIKSNSEAQRDYALTIIQAIVSMIPVFDIILPEVSVPLSMGIIASSMGISFDQLINGDTYEERRSA |
| | location | IPGVATNAVLLGISFALPYLISKASENKVILSQTVSNEDSILNETNIDNFLAENGINKDDIPANGILEVDIKKSGIPVNLVK |
| | domain | ISDEDNQIVAVRGSSQSGIYYEVDIETGYEILSRRVYRTEYNNEIFWIRNGGLKGGQPFDFENLDIPTFFVDKPYSELASS |
| | | PELSFINDDSPLLFPYVDSRLPKPTSEMDISYYSSNFSSFAENTVTLMRGATEEEAWNIAYYKTAGGSNKELEEIFIGGG |
| | | PQANLSFTEYTSNIRSADAASRRHFLVVINVKIKYISNDNVLYANHWAIPDEAPVEVLA |
| 17 | CNF3 | VVDRRFIFPEPPTPPKLSLIQKISQRFFTEDIDETSRINFQRLNSG |
| | Lu/BCAM | |
| | domain | |
| 18 | CNFY | MKNQWQHQYFLSYSELVANFPSPEKVVSDYIKHKFSTTLPWFGWADPDNLYFIRFTQSRSNNKSYTGWDHLGKYAIE |
| | LRP37kDa | TLTLTQAAIVNIGSRFDIFDEANSTAGIYKTNNADSFDETNEAKMLPSEYLYFLRDCDFSNLYNKALSDYWAENYEKF |
| | domain | STLLQNYYISSAYYLYKDSAISKDEYEFSIDAIFNKKSKILRYYFDVYGYYSSDMFVAMNDNKTMLFIP |
| 19 | CNFY | GATNPFIFADNITDLRDKIKALISDKNTRELFSKHFSLYDRQDGNTYLGVNSMLEQIVSGVVDTNYIMYSNKNIRERNV |
| | trans- | FGSMAFSTRERSFNDGDVIIKSNAEVQRDYALNVLQTILSLSPIFDIVLPEVSIPISLGITASSVGISFDELINGDTYEERRS |
| | location | AIPGLATNTVLLGISFAIPFLISKAEENKLIINNLVGSDENILNKNNLGDFLEKYNISESDIPENGSLVINLKNTNVPVRLV |
| | domain | KLNDEEGEIVAIKGSTLSGIYYEVDTETGYEILSRRVFRTEYNEKIYWTRGGGLKGGQPFNFEGLDIPVYFIDKPYSELA |
| | | SSVELSFVNDDSPLLFPEMDSRLPKPTPELDIKYYSSNLSSFKEDTVILMRGTTEEEAWNIANYKTAGGSNKDLEENFIE |
| | | AGPQFNLSFSEYTSSINSADTASRKHFLVIIKVQVKYISNDNVLYANHWAIPDEAPVEVLA |
| 20 | CPP from | MGNQWQQKYLLEYNELVSNFPSPERVVSDYIKNCFKTDLPWFSRIDPDNAYFICFSQNRSNSRSYTGWDHLGKYKTE |
| | CNF1 | VLTLTQAALINIGYRFDVFDDANSSTGIYKTKSADVFNEENEEKMLPSEYLHFLQKCDFAGVYGKTLSDYWSKYYDK |
| | | FKLLLKNYYISSALYLYKNGELDEREYNFSMNALNRSDNISLLFFDIYGYYASDIFVAKNNDKVMLFIPGAKKPFLFKK |
| | | NIADLRLTLKELIKDSDKQQLLSQHFSLYSRQDGVSYAGVNSVLHAIENDGNFNESYFLYSNKTLSNKDVFDAIAISVK |
| | | KRSFSDGDIVIKSNSEAQRDYALTILQTILSMTPIFDIVVPEVSVPLGLGIITSSMGISFDQLINGDTYEERRSAIPGLATNA |
| | | VLLGLSFAIPLLISKAGINQEVLSSVINNEGRTLNETNIDIFLKEYGIAEDSISSTNLLDVKLKSSGQHVNIVKLSDEDNQI |
| | | VAVKGSSLSGIYYEVDIETGYEILSRRIYRTEYNNEILWTRGGGLKGGQPFDFESLNIPVFFKDEPYSAVTGSPLSFINDD |
| | | SSLLYPDTNPKLPQPTSEMDIVNYVKGSGSFGDRFVTLMRGATEEEAWNIASYHTAGGSTEELHEILLGQGPQSSLGFT |
| | | EYTSNVNSADAASRRHFLVVIKVHVKYITNNNVSYVNHWAIPDEAPVEVLAVVDRRFNFPEPSTPPDISTIRKLLSLRY |
| | | FKESIESTSKSNFQKLSRGNIDVLKG |
| 22 | CPP from | MNVQWQQKYLLEYNELVSNFPSPERVVSDYIRRCFKTDLPWFSQVDPDNTYFIRFSQSRSNSRSYTGWDHLGKYKTG |
| | CNF2 | VLTLTQAALINIGYHFDVFDDANASAGIYKTSSADMFNEKNEEKMLPSEYLYFLKGCDFSGIYGRFLSDYWSKYYDKF |
| | | KLLLKNYYISSALYLYKNGEIDEYEYNFSISALNRRDNISLFFFDIYGYYSSDMFVAKNNERVMLFIPGAKKPFLFEKNI |
| | | ADLRISLKNLIKENDNKQLLSQHFSLYSRQDGITYAGVNSVLNAIENDGVFNESYFLYSNKRINNKDVFDAVAFSVKK |
| | | RSFSDGDIVIKSNSEAQRDYALTILQTILSMTPIFDVAIPEVSVTLGLGIIASSMGISFDQLINGDTYEERRSAIPGLATNA |
| | | ALLGLSFAIPFLISKAGTNQKILSRYTKHEIRTLNETNIDMFLEEYGINKNSISETKVLEVELKGSGQHVNIVKLSDEDSK |
| | | IVAVKGNSLSGIYYEVDIETGYEISSRRIYRTEYNDKIFWTRGGGLKGGQSFDFESLKLPIFFKDEPYSAVPGSSLSFIND |
| | | DSSLLYPNSTPKLPQPTPEMEIVNYVKRAGDFGERLVTLMRGTTEEEAWNIARYHTAGGSTEELHEILLGQGPQSSLGF |
| | | TEYTSNINSADAASRRHFLVVIKVQVKYINNNNVSHVNHWAIPDEAPVEVLAVVDRRFNFPEPSTPPNISIIHKLLSLRY |
| | | FKENIESTSRLNLQKLNRGNIDIFKG |
| 24 | CPP from | MNTQWQQKYLLEYNDLVSKFPSPEKVTSDYIKHKFKTDLPWFSRVDPDKTYFIQFSQNRSNSRSYTGWDHLGKYKT |
| | CNF3 | DALTLTQAAIINIGYRFEVFDEANATAGIYTTNNADLFDETNEAKMLPSEYLYFLKNCDFAGLYNKALSDYWSKNHE |
| | | KFKLLLKNYYISSSLYLYKNNVISKDEHEFTMKALNRDDNIELFSFDIYGYYSSDIFGAKNDDRIMLFIPGATNPFLFSE |
| | | NISHLRTHLKELIKENDNRELLSRHFSLYDCQDGSTFYGVDSVLKEIVNGNFNESYFMYTYKKFNERDVFDAISFSVQK |
| | | RSFSDGDTIIKSNSEAQRDYALTIIQAIVSMIPVFDIILPEVSVPLSMGIIASSMGISFDQLINGDTYEERRSAIPGVATNAV |
| | | LLGISFALPYLISKASENKVILSQTVSNEDSILNETNIDNFLAENGINKDDIPANGILEVDIKKSGIPVNLVKISDEDNQIV |
| | | AVRGSSQSGIYYEVDIETGYEILSRRVYRTEYNNEIFWIRNGGLKGGQPFDFENLDIPTFFVDKPYSELASSPELSFINDD |
| | | SPLLFPYVDSRLPKPTSEMDISYYSSNFSSFAENTVTLMRGATEEEAWNIAYYKTAGGSNKELEEIFIGGGPQANLSFTE |
| | | YTSNIRSADAASRRHFLVVINVKIKYISNDNVLYANHWAIPDEAPVEVLAVVDRRFIFPEPPTPPKLSLIQKISQRFFTED |
| | | IDETSRINFQRLNSGNINVLKGR |
| 26 | CPP from | MKNQWQHQYFLSYSELVANFPSPEKVVSDYIKHKFSTTLPWFGWADPDNLYFIRFTQSRSNNKSYTGWDHLGKYAIE |
| | CNFY | TLTLTQAAIVNIGSRFDIFDEANSTAGIYKTNNADSFDETNEAKMLPSEYLYFLRDCDFSNLYNKALSDYWAENYEKF |
| | | STLLQNYYISSAYYLYKDSAISKDEYEFSIDAIFNKKSKILRYYFDVYGYYSSDMFVAMNDNKTMLFIPGATNPFIFAD |
| | | NITDLRDKIKALISDKNTRELFSKHFSLYDRQDGNTYLGVNSMLEQIVSGVVDTNYIMYSNKNIRERNVFGSMAFSTR |
| | | ERSFNDGDVIIKSNAEVQRDYALNVLQTILSLSPIFDIVLPEVSIPISLGITASSVGISFDELINGDTYEERRSAIPGLATNT |
| | | VLLGISFAIPFLISKAEENKLIINNLVGSDENILNKNNLGDFLEKYNISESDIPENGSLVINLKNTNVPVRLVKLNDEEGEI |
| | | VAIKGSTLSGIYYEVDTETGYEILSRRVFRTEYNEKIYWTRGGGLKGGQPFNFEGLDIPVYFIDKPYSELASSVELSFVN |
| | | DDSPLLFPEMDSRLPKPTPELDIKYYSSNLSSFKEDTVILMRGTTEEEAWNIANYKTAGGSNKDLEENFIEAGPQFNLSF |
| | | SEYTSSINSADTASRKHFLVIIKVQVKYISNDNVLYANHWAIPDEAPVEVLAVVDRRFIFPEPPVKPKLSFIQKIANRFLT |
| | | ENVAEISSINFRRLNSGNINVLKG |

1-15. (canceled)

16. A cell-penetrating polypeptide (CPP), which comprises an amino acid sequence that is derived from, or consists of, a portion of a virulence factor, wherein said CPP is associated, or suitable to be associated, to a heterologous cargo.

17. The CPP according to claim 16, wherein said virulence factor is a Cytotoxic Necrotizing Factor (CNF) family member CNF member chosen among Cytotoxic Necrotizing Factor 1 (CNF1), Cytotoxic Necrotizing Factor 2 (CNF2), Cytotoxic Necrotizing Factor 3 (CNF3), and Cytotoxic Necrotizing Factor γ (CNFγ).

18. The CPP according to claim 16, wherein said CPP comprises from N-terminus to C-terminus:
a first receptor binding domain of a virulence factor, and
one translocation domain of a virulence factor.

19. The CPP according to claim 16, wherein said first receptor binding domain is a Laminin Receptor binding domain.

20. The CPP according to claim 19, wherein said Laminin Receptor binding domain comprises or consists of an amino acid sequence that is at least 85% identical to SEQ ID NO: 9, SEQ ID NO: 12, SEQ ID NO: 15 or SEQ ID NO: 18.

21. The CPP according to claim 18, wherein said translocation domain comprises or consists of an amino acid sequence that is at least 85% identical to SEQ ID NO: 10, SEQ ID NO: 13, SEQ ID NO: 16 or SEQ ID NO: 19.

22. The CPP according to claim 18, wherein said CPP further comprises a second receptor binding domain.

23. The CPP according to claim 22, wherein said second receptor binding domain comprises or consists of an amino acid sequence that is at least 85% identical to SEQ ID NO: 11, SEQ ID NO: 14 or SEQ ID NO: 17.

24. The CPP according to claim 16, wherein said CPP comprises or consists of an amino acid sequence that is at least 85% identical to SEQ ID NO: 20, SEQ ID NO: 22, SEQ ID NO: 24 or SEQ ID NO: 26.

25. A complex comprising the cell-penetrating polypeptide (CPP) according to claim 16 associated to a cargo.

26. A polynucleotide or a set of polynucleotides encoding the cell-penetrating polypeptide (CPP) according to claim 16, or encoding the CPP and encoding and/or comprising the heterologous cargo or a portion of the heterologous cargo.

27. A recombinant expression vector comprising the polynucleotide or set of polynucleotides of claim 26.

28. A cell comprising a cell-penetrating polypeptide (CPP) according to claim 16, or a complex comprising the CPP associated to a cargo, or a polynucleotide or set of polynucleotides encoding the CPP, or encoding the CPP and encoding and/or comprising the heterologous cargo or a portion of the heterologous cargo, or a recombinant expression vector comprising said polynucleotide or set of polynucleotides.

29. An in vitro method for directing or delivering a molecule of interest into a cell comprising contacting the cell with the complex comprising the CPP according to claim 16 associated to a cargo, with a polynucleotide or set of polynucleotides encoding the CPP, or encoding the CPP and encoding and/or comprising the heterologous cargo or a portion of the heterologous cargo, or with a recombinant expression vector comprising said polynucleotide or set of polynucleotides.

30. A method of treating a subject in need thereof, comprising administering to said subject a therapeutically effective amount of a cell-penetrating polypeptide (CPP) according to claim 16, or a complex comprising the CPP associated to a cargo, or a polynucleotide or set of polynucleotides encoding the CPP, or encoding the CPP and encoding and/or comprising the heterologous cargo or a portion of the heterologous cargo, or a recombinant expression vector comprising said polynucleotide or set of polynucleotides.
